(19) United States
(12) Patent Application Publication
(10) Pub. No.: US 2010/0152591 A1
(43) Pub. Date: Jun. 17, 2010

(54) APPARATUS AND METHOD FOR NON-INVASIVE AND MINIMALLY-INVASIVE SENSING OF PARAMETERS RELATING TO BLOOD

(76) Inventors: Zengpin Yu, Palo Alto, CA (US); Butrus T. Khuri-Yakub, Palo Alto, CA (US); Xuefeng Cheng, Cupertino, CA (US); Daniel Hwan Kim, Mountain View, CA (US)

Correspondence Address: PATTERSON & SHERIDAN, L.L.P., 3040 POST OAK BOULEVARD, SUITE 1500, HOUSTON, TX, 77056, US

(21) Appl. No.: 12/618,614

(22) Filed: Nov. 13, 2009

Related U.S. Application Data
(63) Continuation of application No. 11/259,858, filed on Oct. 26, 2005, now abandoned.

(51) Int. Cl. A61B 5/02

(52) U.S. Cl. 600/481

(57) ABSTRACT

A system and method for monitoring one or more parameters relating to blood, such as cardiac output, of a patient is provided. The system preferably includes an acoustic energy transducer unit configured and positioned to transmit acoustic energy into a target structure, preferably a blood vessel, within the patient so as to induce a measurable change, preferably a change in blood volume, within the target structure. The transducer unit can be an ultrasonic array, annular array, or groups thereof, or a single element transducer. The unit can also be a vibrator or acoustic loudspeaker. An optical transmitter transmits light into the target structure, and an optical receiver senses light scattered from within the target structure. The blood parameter can then be estimated from the sensed scattered radiation. Relative blood oxygen saturation in the blood vessel can be estimated by transmitting two wavelengths to measure oxy-hemoglobin and deoxy-hemoglobin.

### CROSS-REFERENCE TO RELATED APPLICATION

This disclosure is related to co-pending U.S. patent application Ser. No. 11/095,091, filed 30 Mar. 2005, in the name of John F. Black, Daniel Hwan Kim, and Butrus T. Khuri-Yakub, entitled, “Apparatus and Method for Non-Invasive and Minimally-Invasive Sensing of Venous Oxygen Saturation and pH Levels,” and to co-pending U.S. patent application Ser. No. 11/233,308, filed 22 Sep. 2005, in the name of Xuefeng Cheng, Daniel Hwan Kim, and Butrus T. Khuri-Yakub, entitled, “Apparatus and Method for Non-Invasive and Minimally-Invasive Sensing of Parameters Relating to Blood,” both which are hereby incorporated by reference as if fully set forth herein.

### FIELD

This disclosure is related to techniques for monitoring vital bodily functions, including cardiac output. It relates in particular to methods and apparatus for non-invasive and minimally-invasive real-time monitoring of parameters such as venous oxygenation saturation or pH in a vessel, an organ or tissue containing blood.

### BACKGROUND

Cardiac output is defined as the volume of blood circulated per minute. It is equal to the heart rate multiplied by the stroke volume (the amount ejected by the heart with each contraction). Cardiac output averages approximately 5 liters per minute for an average adult at rest, although it may reach up to 30 liters/minute during extreme exercise.

Cardiac output is of central importance in the monitoring of cardiovascular health, as discussed by Conway, “Clinical assessment of cardiac output,” Eur. Heart J., 11, 148-150 (1990). Accurate clinical assessment of the circulatory status is particularly desirable in critically ill patients in the ICU and patients undergoing cardiac, thoracic, or vascular interventions, and has proven valuable in long term follow-up of outpatient therapies. As the patient's hemodynamic status may change rapidly, continuous monitoring of cardiac output will provide information allowing rapid adjustment of therapy. Measurements of cardiac output and blood pressure can also be used to calculate peripheral resistance.

A recent review of the various techniques for measuring cardiac output is given in Linton and Gilon, “Advances in non-invasive cardiac output monitoring,” Annals of Cardiac Anaesthesia, 2002, volume 5, p 141-148. This article lists both non/minimally invasive and invasive methods and compares the advantages and disadvantages of each.

The pulmonary artery catheter (PAC) thermodilution method is generally accepted as the clinical standard for monitoring cardiac output, to which all other methods are compared as discussed by Conway and Lund-Johansen (“Thermodilution method for measuring cardiac output,” Europ. Heart J., 11 (Suppl1), 17-20 (1990)). The long history of use has defined the technology, suitable clinical applications, and its inadequacies. Many new methods have attempted to replace the thermodilution technique, but none have so far gained acceptance.

Jansen (J. R. C. Jansen, “Novel methods of invasive/non-invasive cardiac output monitoring,” Abstracts of the 7^{th} annual meeting of the European Society for Intravenous Anesthesia, Lisbon 2004) describes eight desirable characteristics for cardiac output monitoring techniques; accuracy, reproducibility or precision, fast response time, operator independency, ease of use, continuous use, cost effectiveness, and no increased mortality and morbidity. A brief description of some of these techniques follows.

Indicator dilution techniques. There are several indicator dilution techniques including transpulmonary thermodilution (also known as PiCCO technology, from Pulsion Medical Technologies of Munich, Germany), transpulmonary lithium dilution method (LiDCO Group plc of London, UK), PAC based thermodilution and other methods (Vigilance, Baxter; Opti-Q, Abbott; and TruCCOMS, AorTech). U.S. Pat. No. 6,757,544 to Rubinstein et al. teaches the technique of optically monitoring indicator dilution in a non-invasive manner for the purpose of computation of cardiac output, cardiac index, and blood volume. Transpulmonary indicator dilution methods with bolus injections are variations on the conventional bolus thermodilution method. CO is calculated with use of the Steward-Hamilton equation (Geddes, “Cardiac output using the saline dilution impedance technique,” IEEE Engineering in Medicine and Biology magazine, March 1989, 22-26). Application of this equation assumes three major conditions; complete mixing of blood and indicator, no loss of indicator between place of injection and place of detection, and constant blood flow. The errors associated with indicator dilution techniques are primarily related to the violation of these conditions, as discussed by Lund-Johansen (“The dye dilution method for measurement of cardiac output,” Europ. Heart J., 11 (Suppl 1), 6-12 (1990)) and de Leeuw and Birkenhager (“Some comments of the usefulness of measuring cardiac output by dye dilution,” Europ. Heart J., 11 (Suppl 1), 13-16 (1990)). Of the mentioned methods, the transpulmonary indicator dilution methods as well as the so-called ‘continuous cardiac output’ thermodilution methods have been partially accepted in clinical practice as described in, for example, Rödig et al., “Continuous cardiac output measurement: pulse contour versus thermodilution technique in cardiac surgical patients,” Br J Anaesth, 1999; 50: 525.

Fick principle. The direct oxygen Fick approach is currently the standard reference technique for cardiac output measurement, as discussed by Keinanen et al., “Continuous measurement of cardiac output by the Fick principle: Clinical validation in intensive care,” Crit. Care Med 20(3), 360-365 (1992), and Doi et al., “Frequently repeated Fick cardiac output measurements during anesthesia,” J. Clin. Monit., 6, 107-112 (1990). It is generally considered the most accurate method currently available, although there are many possibilities of introducing errors, and considerable care may be needed. However when using the Fick method to trend cardiac output over a short time interval, i.e., during an operation or in an intensive care unit stay, many of these sources of errors are no longer pertinent. The NICO (Novametrix) system is a non-invasive device that applies Fick's principle on CO₂ and relies solely on airway gas measurement as described by Botero et al., “Measurement of cardiac output before and after cardiopulmonary bypass: Comparison among aortic transit-time ultrasound, thermodilution, and noninvasive partial CO₂ rebreathing,” J. Cardiothoracic. Vase. Anesth., 18(5) 563-572 (2004). The method calculates effective lung perfusion, i.e., that part of the pulmonary capillary blood flow that has passed through the ventilated parts of the lung. The effects of unknown ventilation/perfusion inequality in patients may explain why the performance of this method shows a lack of agreement between thermodilution and CO₂-rebreathing cardiac output as described in Nielsson et al., “Lack of agreement between thermodilution and CO₂-rebreathing cardiac output,” Acta Anaesthesiol Scand, 2001; 45:680.

Bio-Impedance and condition techniques. The bio-impedance method was developed as a simple, low-cost method that gives information about the cardiovascular system and/or (de)-hydration status of the body in a non-invasive way. Over the years, a diversity of thoracic impedance measurement systems has also appeared. These systems determine CO on a beat-to-beat time base. Studies have been reported with mostly poor results, but in exceptional cases good correlations compared to a reference method. Many of these studies refer to the poor physical principles of the thoracic impedance method as described in Patterson “Fundamentals of impedance cardiography,” IEEE Engineering in Medicine and Biology, 1989; 35 to explain the discrepancies. The accuracy of this technique is increased when the electrodes are placed directly in the left ventricle, rather than on the chest, however this also increases its invasiveness.

Echo Doppler ultrasound. This technique uses ultrasound and the Doppler effect to measure cardiac output. The blood velocity through the aorta causes a ‘Doppler shift’ in the frequency of the returning ultrasound waves. Echo-Doppler probes positioned inside the esophagus with their echo window on the thoracic aorta may be used for measuring aortic flow velocity, as discussed by Schmidlin et al., “Transoesophageal echocardiography in cardiac and vascular surgery: implications and observer variability,” Brit. J. Anaesth., 86(4), 497-505 (2001). Aortic cross sectional area is assumed in devices such as the CardioQ, made by Deltex Medical PLC, Chichester, UK) or measured simultaneously as for example in the HemoSonic device made by Arrow International. With these minimally invasive techniques, what is measured is aortic blood flow, not cardiac output. A fixed relationship between aortic blood flow and cardiac output is assumed. CO can therefore be calculated using this relationship. Abrupt changes in cardiac output are better followed with Doppler systems than with the PAC based continuous cardiac output systems as described in Roeck et al., “Change in stroke volume in response to fluid challenge: assessment using esophageal Doppler,” Intensive Care Med, 2003; 29:1729. This measurement requires an above average level of skill on the part of the operator of the ultrasound machine to get accurate, reliable results.

Arterial pulse contour analysis. The estimation of cardiac output based on pulse contour analysis is an indirect method, since cardiac output is not measured directly but is computed from a pressure pulsation on basis of a criterion or model. The origin of the pulse contour method for estimation of beat-to-beat stroke volume goes back to the Windkessel model as described in, for example, Manning et al., “Validity and reliability of diastolic pulse contour analysis (Windkessel model) in humans,” Hypertension, 2002 May; 39(5):963-8. Most pulse contour methods are based on this model explicitly or implicitly as described in Rauch et al., “Pulse contour analysis versus thermodilution in cardiac surgery,” Acta Anaesthesiol Scand, 2002; 46:424, Linton et al., “Estimation of changes in cardiac output from arterial blood pressure waveform in the upper limb,” Sr J Anaesth, 2001; 86:486 and Jansen et al., “A comparison of cardiac output derived from the arterial pressure wave against thermodilution in cardiac surgery patients,” Sr J Anaesth, 2001; 87:212.

Arterial pulse contour analysis techniques relate an arterial pressure or pressure difference to a flow or volume change. Three pulse contour methods are currently available; PiCCO (Pulsion), PulseCO (LiDCO) and Modelflow (TNO/BMI). All three of these pulse contour methods use an invasively measured arterial blood pressure and they should be calibrated. PiCCO is calibrated by transpulmonary thermodilution, LiDCO by transpulmonary lithium dilution and Modelflow by the mean of 3 or 4 conventional thermodilution measurements equally spread over the ventilatory cycle. Output of these pulse contour systems is calculated on a beat-to-beat basis, but presentation of the data is typically within a 30-second window. A non-invasive pulse contour development is the combination of non-invasively measured arterial finger blood pressure with Modelflow as described in Hirschl et al., “Noninvasive assessment of cardiac output in critically ill patients by analysis of finger blood pressure waveform,” Crit. Care Med, 1997; 25:1909.

None of the above-mentioned CO techniques combines all of the eight “Jansen” criteria mentioned above. With respect to accuracy and precision, a number of methods may approach the thermodilution method with a precision of 15%. None of these new techniques has displaced conventional thermodilution based on the averaged result of 3 or 4 measurements done equally spread over the ventilatory cycle as described in Jansen et al., “An adequate strategy for the thermodilution technique in patients during mechanical ventilation,” Intensive Care Med, 1990; 16:422. Under research conditions, the use of this conventional thermodilution method remains the method of choice. However, in clinical settings, the lower precision of the continuous cardiac output techniques may be outweighed by their advantages of being automatic and continuous.

In addition to measuring cardiac output, it is also desirable in many critical care situations to continuously monitor a patient's blood oxygen level. Currently, hospitals routinely monitor blood oxygenation by pulse oximetry with a monitor attached to the patient's finger or earlobe as described for example in Silva et al., “Near-infrared transmittance pulse oximetry with laser diodes,” J. Biomed., Opt. 8(3), 525-533 (2003). Typically, the oxygen monitor is a pair of light-emitting diodes (LED) and photodiodes on a probe clipped to a part of the patient's body. Red light from the LED reflects from the blood in a part of the patient's body, such as an earlobe or finger-tip. As a patient's oxygenation level drops, the blood becomes more blue, reflecting less red light to the photodiode. Such blood-oxygen monitors customarily measure percent of normal. Reassuring (normal) ranges are from 95 to 100 percent. For a patient breathing room air, at not far above sea level, an estimate of arterial oxygenation can be made from the blood-oxygen monitor reading. Unfortunately, measurements from such oxygen monitors cannot be reliably correlated to oxygenation in the patient's venous blood. Venous oxygen saturation is also a valuable parameter in the diagnosis of septic and cardiogenic shock as described below.

Other methods of measuring oxygenation: Diffuse optical tomography methods as described, for example, in Boas et al., “Method for monitoring venous oxygen saturation,” U.S. Patent publication 2004/0122300 are conceptually appealing but are useful only where the vessels in the vicinity of the diffusing photon field are isolated veins. The presence of mixed arterial and venous blood complicates the problem as described by Wolf et al., “Continuous noninvasive measurement of cerebral arterial and venous oxygen saturation at the bedside in mechanically ventilated neonates,” Crit. Care. Med, 25(9), 1579-1582 (1997).

Ultrasound-tagged optical spectroscopy involves overlapping an ultrasound wave and a diffusing optical field, and modulating the frequency of the probe photons or their trajectories. A number of different technologies have been developed that utilize some interaction between ultrasound radiation and electromagnetic radiation. U.S. Pat. No. 5,212,667 to Tomlinson et al. and U.S. Pat. No. 5,174,298 to Dolfi et al. teach the technique of ultrasound-tagged frequency-modulated imaging. Other patents teaching variations on the theme of frequency-modulated ultrasound tagging techniques include U.S. Pat. No. 6,815,694 to Sfez et al., U.S. Pat. No. 6,738,653 to Sfez et al., U.S. Pat. No. 6,041,248, to Wang, U.S. Pat. No. 6,002,958 to Godik, U.S. Pat. No. 5,951,481 to Evans, U.S. Pat. No. 5,293,873 to Fang. Trajectory modulation is detected by monitoring the speckle pattern of the photons emerging from the target. Image reconstruction techniques are then used to recreate a map of the path the photons followed in the medium. Imaging the speckle resulting from trajectory changes requires significant computation power and post-processing to yield an image. The technique has limited resolution, and is not yet capable of yielding functional (oxygenation) information in a fast flowing vessel.

Some variations of ultrasound-tagged frequency-modulated imaging rely on observing the frequency shift induced by the photoacoustic effect when an electromagnetic wave interacts in a medium with a sound wave. The electromagnetic wave (having a characteristic frequency ω_{OPT}) receives a frequency shift at the ultrasound frequency ω_{US} to either the + or −side of the carrier wave ω_{OPT}. Frequency modulation is detected by measuring the frequency shifted photons by, for example, using a Fabry-Perot etalon as described by Sakadzic and Wang, “High resolution ultrasound modulated optical tomography in biological tissues,” Opt. Lett., 29(23) 2004, P 2770-2772. Since the Doppler shifts induced by the ultrasound wave are very small compared to the probe photon carrier wave frequency, the detection system should be extremely sensitive to small frequency shifts. In addition, the frequency shift can be to both larger and smaller frequency of the initial carrier wave, and therefore some self-cancellation may result.

There is a need in the art to be able to measure venous oxygen saturation levels in various vascular structures in the body, and from this, be able to calculate cardiac output. There is a need to make these measurements non-invasively or with minimal invasiveness. There is a need to be able to make these measurements in an MRI-/CT/X-Ray instrument compatible manner, thus preferably not using ferromagnetic materials in construction, and using designs such that the probe on/in the body may be remotely coupled to the control system away from the magnetic field or ionizing radiation sources generated by the MRI instrument or CT/X-Ray.

There is a need in the art to make these measurements in a manner that does not depend on the melanin content of the skin. There is a need to make these measurements in a manner such that the result may be arrived at in a short time period, i.e., such that extensive post-processing of the data is not required, so that the physician may make accurate timely diagnostic and therapeutic decisions.

### SUMMARY

Many or all of the disadvantages associated with the prior art can be significantly alleviated through embodiments of the present disclosure.

According to some embodiments of the present disclosure, a system for monitoring one or more parameters relating to blood, such as cardiac output, of a patient is provided. The system preferably includes an acoustic energy transducer unit configured and positioned to transmit acoustic energy into a target structure, preferably a blood vessel, within the patient so as to induce a measurable change, preferably a change in blood volume, within the target structure. At least one optical transmitter is configured to generate electromagnetic radiation containing photons having a specific interaction with at least one target chromophore in the target structure. The transmitter is configured and positioned to transmit the radiation into the target structure. At least one optical receiver is configured and positioned to detect a portion of the electromagnetic radiation scattered from within the target structure. A processor is adapted to estimate the parameter relating to the patient's blood, with the estimation being based in part on the scattered radiation detected from within the target structure, and preferably also on the measured induced change within the target structure. The optical transmitter can be configured to transmit continuous wave or pulsed electromagnetic radiation into the target structure.

The system preferably uses at least one ultrasound transducer to provide an ultrasound radiation pressure field into the target structure so as to modulate the target structure at a modulation frequency, and a filter to select detected electromagnetic radiation having a modulation component at the same frequency as the modulation frequency, or at a harmonic of the modulation frequency. The transducer unit can be in the form of a linear array, a group of linear arrays, a single element transducer, an annular array transducer or groups of annular array transducers. If a single element or annual array transducer is provided, an adapter may be used to allow movement of the transducer with respect to the patient's target structure. The ultrasound radiation pressure field preferably induces changes in the shape of the target structure which induces a change in the blood flow in the target structure.

The transducer unit can take the form of a vibrator or acoustic loudspeaker adapted and positioned to transmit vibrational or sonic energy into the target structure thereby inducing a change in blood flow in the target structure. An ultrasound transducer can also be adapted to generate an image of tissues including the target structure to enable placement of the optical transmitter and optical receiver on the patient so as to enhance the accuracy of the monitoring of the system.

The optical transmitters can also be configured and positioned to transmit the radiation into a second area to estimate absorption properties with the second area thereby increasing the accuracy of the measurement of the blood parameters. The system preferably calculates relative blood oxygen saturation in the blood vessel, by transmitting two wavelengths to measure oxy-hemoglobin and deoxy-hemoglobin. The target structure can be, for example, the patient's internal jugular vein. The acoustic energy transducer unit, optical transmitter and receiver can be partially mounted on a sensor patch designed to be engaged to the patient's skin.

The present disclosure is also embodied in a method for monitoring one or more parameters relating to blood of a patient comprising the steps of inducing a change in blood volume in a target structure within the patient; transmitting two or more frequencies of electromagnetic radiation into the target structure; sensing the two or more frequencies of electromagnetic radiation having scattered from within the target structure; and calculating the one or more parameters relating to blood based at least in part on the sensed electromagnetic radiation.

### BRIEF DESCRIPTION OF THE DRAWINGS

The teachings of the present disclosure can be readily understood by considering the following detailed description in conjunction with the accompanying drawings, in which:

FIG. 1 is a schematic view of an embedded vascular structure that is an example of a suitable target for measurement with embodiments of the present disclosure.

FIG. 2A is a schematic diagram of an apparatus according to an embodiment of the present disclosure.

FIG. 2B is a close-up cross-sectional schematic diagram illustrating an example of use of the apparatus of FIG. 2A

FIG. 3 is a schematic diagram of a three-wavelength pulsed optical source for use in embodiments of the present disclosure.

FIG. 4 is a schematic diagram of an all-electronic optical source for use in embodiments of the present disclosure.

FIG. 5 is an example of a source of three wavelengths using an Optical Parametric Oscillator for use with embodiments of the present disclosure.

FIG. 6 is a schematic diagram illustrating an example of signal broadening expected at a tissue boundary.

FIG. 7 is a schematic diagram of an apparatus using the principle of time gated upconversion according an alternative embodiment of the present disclosure.

FIG. 8 is a schematic diagram of an apparatus having two pulsed optical sources according to another alternative embodiment of the present disclosure proposed implementation of the present disclosure.

FIG. 9A is a schematic diagram depicting time-gated upconversion detector that can be used in the apparatus of FIG. 8.

FIG. 9B is a schematic diagram depicting an alternative time-gated upconversion detector that can be used in the apparatus of FIG. 8.

FIG. 10 is a schematic diagram depicting a second apparatus having a background-free time-gated upconversion detector according to another embodiment of the present disclosure.

FIG. 11 is a graph of the absorption of oxy-hemoglobin and water in the range 700-1200 nm, an expected variation of the scattering coefficient as a function of wavelength, and an expected difference between an artery with fully oxygen-saturated blood and a vein where the oxygen saturation is 55%.

FIGS. 12A-12B are schematic diagrams of sensors that can be used with embodiments of the present disclosure.

FIG. 12C is a three-dimensional diagram of an alternative sensor according to an embodiment of the present disclosure.

FIG. 12D is a cross-sectional diagram taken along line D-D of FIG. 12C.

FIG. 13 is a schematic diagram illustrating an example of trans-dermal measurement of oxygenation of blood in the internal or external jugular veins.

FIG. 14 is a schematic diagram of a portion of the circulatory system showing examples of locations that may be probed for blood oxygenation using embodiments of the present disclosure.

FIG. 15 is a horizontal cross-section through the chest showing examples of of locations that may be probed for blood oxygenation using embodiments of the present disclosure.

FIG. 16 is a close-up vertical thoracic cross-section illustrating a sensor placed in the left bronchus to probe oxygenation of the left pulmonary artery and descending thoracic aorta.

FIG. 17 is a schematic thoracic diagram illustrating an example of trans-tracheal placement of a sensor according to an embodiment of the present disclosure.

FIG. 18A is sagittal cross-sectional schematic diagram illustrating a normal heart.

FIG. 18B is a sagittal cross-sectional schematic diagram illustrating a heart exhibiting Patent Ductus Arteriosus (PDA).

FIG. 18C is a sagittal cross-sectional schematic diagram illustrating a heart exhibiting Patent Foramen Ovale (PFO).

FIG. 19 is a thoracic axial cross-sectional schematic diagram illustrating examples of sensor placement for cardiac mapping in newborn infants according to an embodiment of the disclosure.

FIG. 20 is a sagittal cross-sectional schematic diagram illustrating examples of sensor placement for monitoring of fetal blood oxygenation.

FIGS. 21A-B are a transmit array and an associated transmit time delay profile according to an embodiment of the disclosure.

FIG. 22 is an illustration of a focal area of a focused ultrasonic beam, according to embodiments of the disclosure.

FIGS. 23A-B are a multiple beam focusing array and associated time delay profile, according to embodiments of the disclosure.

FIG. 24 shows a multi-beam focal area in greater detail, according to embodiments of the disclosure.

FIG. 25 is a two-dimensional transducer array, according to embodiments of the disclosure.

FIGS. 26A-B show a single element transducer having a circular cross-section, according to a preferred embodiment of the disclosure.

FIGS. 27 A-C show an annular array transducer according to embodiments of the disclosure.

FIGS. 28A-C show a transducer adapter, according to embodiments of the disclosure.

FIG. 29 is a ring array transducer, according to embodiments of the disclosure.

FIGS. 30A-B show a grouped arrangement of annular transducers, according to embodiments of the disclosure.

FIG. 31 is a transducer array group having non-parallel transducers, according to embodiments of the disclosure.

FIG. 32 is a mechanical vibrator arrangement according to embodiments of the disclosure.

FIG. 33 shows placement of a vibrator group on the skin of a patient, according to embodiments of the disclosure.

FIGS. 34A-B show audio loudspeaker arrangement for generating vibrational energy in target structures, according to embodiments of the disclosure.

FIG. 35 shows a system for making relative measurements relating to blood oxygenation according to an embodiment of the disclosure.

FIGS. 36A and 36B show a sensor/transducer unit according to embodiments of the disclosure.

FIG. 37 is a flowchart illustrating several steps relating to measuring cardiac output according to embodiments of the disclosure.

### DESCRIPTION OF THE SPECIFIC EMBODIMENTS

Although the following detailed description contains many specific details for the purposes of illustration, anyone of ordinary skill in the art will appreciate that many variations and alterations to the following details are within the scope of the disclosure. Accordingly, the exemplary embodiments of the disclosure described below are set forth without any loss of generality to, and without imposing limitations upon, the claims which follow thereafter.

### GLOSSARY

As used herein, the following terms have the following meanings:

Acoustic and Acoustic Energy: refers to all frequencies including sub-sonic, vibrations, sonic, and ultrasonic.

Continuous wave (CW) laser: A laser that emits radiation continuously rather than in short bursts, as in a pulsed laser.

Diode Laser: Refers to a light-emitting diode designed to use stimulated emission to generate a coherent light output. Diode lasers are also known as laser diodes or semiconductor lasers. A diode-pumped laser refers to a laser having a gain medium that is pumped by a diode laser.

Mode locked laser: A laser that emits radiation in short bursts, as in a pulsed laser. Typically these pulses are on the order of 0.1-100 picoseconds in temporal length and preferably 1-50 picoseconds.

Highly Non-linear Fiber: A fiber characterized by having a guiding core with properties that can be used to convert electromagnetic radiation at one frequency to another provided there is sufficient intensity at the originating frequency and the fiber has sufficient length.

Upconversion Process: A process by which photons of a given frequency are converted to photons of shorter wavelength (higher frequency). This technique may be used, e.g., to bring infra-red photons into the detection range of silicon detectors, for example, or may be used in a pulsed configuration to give temporal selectivity in which photons are upconverted and hence detected.

Non-Linear Crystal: A crystal made of a material having special optical properties allowing the frequency of an incoming electromagnetic wave to be shifted according to predictable rules and conditions.

Optical Parametric Oscillator: A process by which a photon at a pump frequency ω_{P} is converted in a material inside a resonator to two photons of lower frequency, typically called the signal and idler photons with the relationship:

ω_{P}=ω_{SIG}+ω_{IDL}

Optical Parametric Amplifier: A process by which a photon at a pump frequency ωₚ is converted in a material (but without the need for an external resonator) to two photons of lower frequency, typically called the signal and idler photons with the relationship:

ωₚ=ω_{sig}+ω_{idl}

As stated above, there are eight desirable characteristics for cardiac output (CO) monitoring techniques: accuracy, reproducibility or precision, fast response time, operator independency, ease of use, continuous use, cost effectiveness, and no increased mortality and morbidity associated with its use. None of the present CO monitoring techniques satisfactorily combines all eight criteria mentioned above. The Fick principle involves measuring the oxygen consumption (V0₂) per minute (e.g., using a spirometer), measuring the oxygen saturation of arterial blood using for example standard pulse oximetry on the finger, and measuring venous oxygen saturation in the pulmonary artery or superior vena cava.

From these values, one can calculate:

$$CardiacOutput = \frac{Oxygen\_Consumption}{( {ArterialSa O}_{2} - {VenousSa O}_{2} \times [ Hb ] \times 1.36}$$

where Arterial Sa0₂ and Venous Sa0₂ are respectively the arterial and venous oxygen saturation, [Hb] is the blood hemoglobin concentration and 1.36 is a factor subsuming the oxygen carrying capacity of the hemoglobin. [Hb] can be related simply to the hematocrit (Hct), a routinely measured parameter defined as the percent of whole blood that is composed of red blood cells (erythrocyte volume to total volume expressed as a percentage). The range for Hct is 32-50% in “normal” “healthy” people. Hct does not tend to change dramatically and quickly (unless the patient is bleeding severely), so it is sufficient to take a sample every 4-6-8 hours, for example and update the Fick calculation periodically. Hematocrit (hct) can be measured, e.g., by taking a sample of blood and spinning it down in a centrifuge and calculating the volumes.

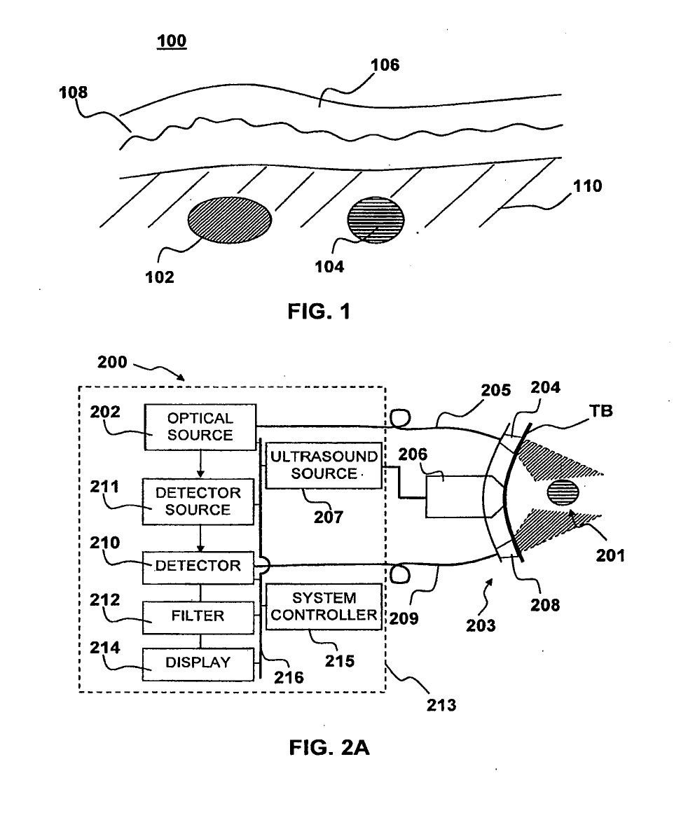

The Fick principle relies on the observation that the total uptake of (or release of) a substance by the peripheral tissues is equal to the product of the blood flow to the peripheral tissues and the arterial-venous concentration difference (gradient) of the substance. In the determination of cardiac output, the substance most commonly measured is the oxygen content of blood, and the venous saturation is measured in the pulmonary artery using a catheter as, for example, described by Powelson et al., “Continuous monitoring of mixed venous oxygen saturation during aortic operations,” Crit. Care Med., 20(3), 332-336 (1992). This gives a simple way to calculate the cardiac output. The drawback of drift associated with this type of catheter has been discussed by Souter et al., “Jugular venous desaturation following cardiac surgery,” Brit. J. Anaesth., 81, 239-241 (1998). It is also highly invasive, incompatible with ambulatory measurement, and poses risks of infection due to vascular system breach (femoral or jugular vessel insertion). The nature of the challenge is illustrated schematically in FIG. 1. An embedded vascular structure of a body 100 includes an artery 102 and vein 104, for example, the internal jugular vein and artery in the neck. The vein 102 and artery 104 are located beneath the epidermis 106 and dermis 108 of the body 100. The vein and artery are embedded in and around subcutaneous structures 110, e.g., fat, muscle, tendon, etc.

Assuming there are no shunts across the cardiac or pulmonary system, the pulmonary blood flow equals the systemic blood flow. Measurement of the arterial and venous oxygen content of blood involves the sampling of blood from the pulmonary artery (low oxygen content) and from the pulmonary vein (high oxygen content). In practice, sampling of peripheral arterial blood is a surrogate for pulmonary venous blood.

Embodiments of the present disclosure allow non-invasive or minimally invasive measurement of venous oxygen saturation at a point where the value trends correctly with a direct pulmonary artery catheter measurement. One can apply the above-described Fick principle to such a measurement thereby enabling measurement of cardiac output in a non- or minimally invasive manner. Embodiments of the present disclosure for measuring venous oxygen saturation can also be made insensitive to the presence of shunts in the heart, such as for example, acquired ventricular septal defects, and as such, offer valuable adjunct information if PAC thermodilution or Fick data are already available. This is the case when the sensor is placed on the internal jugular vein.

The value of the venous oxygen saturation is also a useful adjunct diagnostic parameter in its own right. Patients with low cardiac output tend to have low venous oxygen saturation, for example around 50. This low value results from the increased extraction of oxygen in the body tissues due to the poor perfusion resulting from low flow. However, high mixed venous oxygen saturation with low cardiac output can indicate a significant left-to-right shunt across the heart, such as an acquired ventricular septal defect. Embodiments of the present disclosure where the sensor is placed on the internal jugular will allow a measurement of venous oxygen saturation before the heart and pulmonary system, and thus will be insensitive to the presence of these shunts.

Also, by way of example, a presentation of high cardiac output, high venous oxygen saturation, narrow arterio-venous difference and low peripheral resistance might suggest to the physician to test for septic shock. On the other hand, cardiogenic shock is associated with high peripheral resistance. Thus, measurement of cardiac output can help guide and monitor the administration of drugs such as vasodilators/vasoconstrictors and inotropes.

A number of different technologies have been developed that utilize some interaction between ultrasound radiation and electromagnetic radiation. However, these prior art technologies are all distinguishable from the techniques described herein. For example, embodiments of the present disclosure are superior to standard ultrasound-tagged photon techniques in that they are not limited by the ability of the apparatus to detect very small frequency shifts on the detected photons. U.S. Pat. No. 5,212,667 to Tomlinson et al. and U.S. Pat. No. 5,174,298 to Dolfi et al. teach the technique of ultrasound-tagged frequency-modulated imaging. Other patents teaching variations on the theme of frequency-modulated ultrasound tagging techniques include U.S. Pat. No. 6,815,694 to Sfez et al., U.S. Pat. No. 6,738,653 to Sfez et al., U.S. Pat. No. 6,041,248, to Wang, U.S. Pat. No. 6,002,958 to Godik, U.S. Pat. No. 5,951,481 to Evans, U.S. Pat. No. 5,293,873 to Fang.

Ultrasound-tagged frequency modulated imaging relies on observing the frequency shift induced by the photoacoustic effect when an electromagnetic wave interacts in a medium with a sound wave. The electromagnetic wave (having a characteristic frequency ω_{OPT}) receives a frequency shift at the ultrasound frequency ω_{US} to either the + or −side of the carrier wave ω_{OPT}. Heterodyne or interferometric techniques are then used to decouple the frequency shifted wave from the carrier wave. Implementation of the technique utilizes sophisticated lasers with narrow linewidths and concomitantly long coherence lengths in order to resolve the two frequencies. U.S. Pat. No. 6,002,958 to Godik teaches the study of the amplitude modulation induced on an electromagnetic wave by the ultrasound beam and scanning the ultrasound beam in order to form an image of the absorber.

U.S. Pat. No. 6,264,610 to Zhu teaches the use of ultrasound and near-IR imaging as adjunctive imaging techniques, but does not attempt a physical link between the two techniques.

U.S. Pat. No. 5,452,716 to Clift teaches the use of two-wavelength probing using one wavelength specific to the substance being probed and a reference field characterized by another wavelength. This patent does not teach any form of temporal gating, any form of targeting a structure, or any form of depth control using co-located optical and ultrasound fields.

U.S. Pat. No. 6,445,491 to Sucha et al. and U.S. Pat. No. 5,936,739 to Cameron et al. teach the use of optical parametric processes to amplify signals in imaging systems. Neither of these patents teaches the use of upconversion to produce a signal which is necessarily free from background contamination from for example fluorescence processes or Raman scattering. Neither of the patents teaches the use of the very fast non-linearities found in fiber Optical Parametric Amplifiers to yield time-gated information in a straightforward manner.

U.S. Pat. No. 5,451,785 to Faris teaches the use of upconversion processes in a transillumination imaging system.

U.S. Pat. No. 6,665,557 to Alfano et al. teaches spectroscopic and time-resolved optical methods for imaging tumors in turbid media where time gating of the ballistic and near-ballistic photons is used to improve the reconstruction of the image. The more diffusely scattered photons are rejected in this technique and no attempt is made to localize the interaction using ultrasound.

U.S. Publication No. 2004/0122300 A1 Boas et al., U.S. Publication No. 2004/0116789 to Boas et al., U.S. Pat. No. 6,332,093 to Painchaud et al., U.S. Pat. No. 5,630,423 to Wang et al., U.S. Pat. No. 5,424,843 to Tromberg et al. and U.S. Pat. No. 5,293,873 to Fang teach variations on the theme of Photon Migration Spectroscopy, Photon Migration Imaging (PMI), Diffuse Optical Tomography (DOT), or Diffuse Imaging, where photons from a source diffuse through the target and are detected using detectors placed at various distances from the source launch point. The characteristics of the diffusing photons are interpreted to yield functional and structural information about the medium they have diffused through. No attempt is made to “tag” these photons to localize the region of interaction. No attempt is made to time-gate the detected signal. Embodiments of the present disclosure are superior to Photon Migration Imaging (PMI, DOT, etc.) in that they allow accurate depth and location localization of the target.

Embodiments of the present disclosure are also superior to speckle-based imaging techniques because they are insensitive to the speckle decorrelation time of the tissue being probed. This speckle decorrelation is very fast in larger vascular structures with flowing blood inside, preventing use of speckle-based techniques in the types of vessels the current disclosure aims to address.

Embodiments of the present disclosure can also be designed in such a way as to be insensitive to the presence of epidermal melanin (unlike many of the wavelengths used in PMI/DOT and ultrasound-tagged spectroscopy and imaging). Embodiments of the present disclosure can also be designed in a manner that will not suffer from significant solar or environmental background light contamination.

Embodiments of the present disclosure do not require the development of sophisticated single frequency lasers and interferometric detection techniques. As a result, embodiments of the present disclosure will be simpler to implement and more technologically robust in a clinical setting. Apparatus according to embodiments of the present disclosure can use proven telecommunication-based fiber-based technology to yield a robust, small, and efficient product.

Embodiments of the present disclosure do not require 2-D imaging arrays or cameras (for example CCD cameras), and in particular do not require infra-red detector arrays such as InGaAs CCDs. These devices are cooled to achieve low noise conditions, further complicating the experimental/clinical implementation. Apparatus according to embodiments of the present disclosure can use proven single element silicon detectors which do not need to be cooled and which do not need extensive computational support.

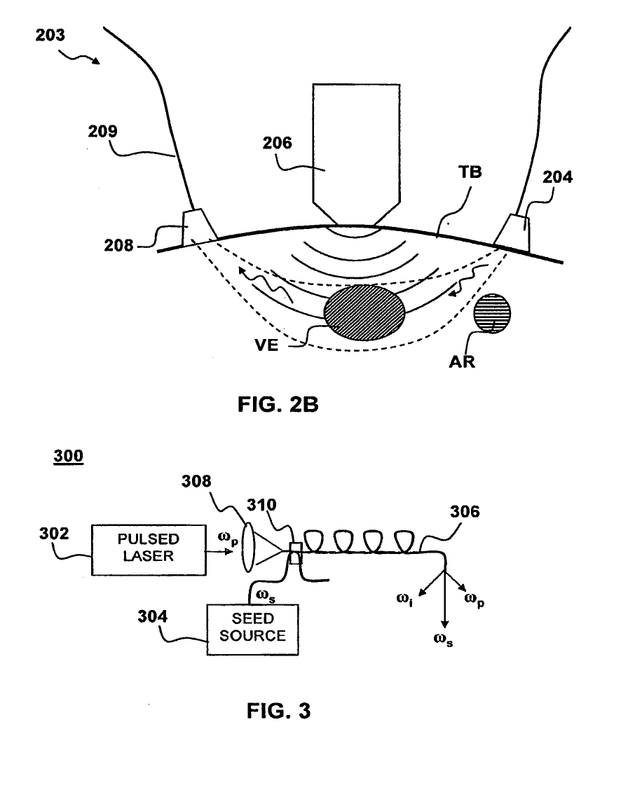

FIG. 2A is a schematic block diagram of a diagnostic apparatus 200 according to an embodiment of the present disclosure. The apparatus 200 generally includes an optical source 202, launch optics 204, an ultrasound transducer 206, collection optics 208, an optical detector 210, associated electronics such as a filter 212 and an optional display 214. The optical source 202 provides pulsed or continuous electromagnetic radiation. The launch optics 204 may include one or more optical fibers 205 that couple the electromagnetic radiation from the optical source 202 to a body 201. Similarly, the collecting optics 208 collect optical signals reflected from within the body 201. The collecting optics 208 may also include one or more optical fibers 209 that couple signals scattered electromagnetic radiation to the optical detector 210. The optical source 202 may supply a timing signal (which may be either optical or electronic) to trigger a detector source 211 that provides an optical signal used in detection of the scattered radiation.

In some embodiments, the launch optics 205, ultrasound transducer 206, and collecting optics may be mounted together in a handpiece to form a combined ultrasound optical sensor 203. In other embodiments, the detector 210 may be part of the sensor 203 without the need for collecting optics. In some embodiments, the optical source 202, optical detector 210, detector source 211, filter 212, display 214 and an ultrasound generator 207 may be part of a remote unit 213 coupled to the sensor 203 by fiberoptics 205, 209 and electrical cables. The remote unit 213 may include a system controller 215. The system controller 215 may include a central processor unit (CPU) and a memory (e.g., RAM, DRAM, ROM, and the like). The controller 215 may also include well-known support circuits, such as input/output (I/O) circuits, power supplies (PIS), a clock (CLK), Field Programmable Gate Arrays (FPGAs) and cache. The controller 215 may optionally include a mass storage device such as a disk drive, CD-ROM drive, tape drive, or the like to store programs and/or data. The controller may also optionally include a user interface unit to facilitate interaction between the controller 215 and a user. The user interface may include a keyboard, mouse, joystick, light pen or other device. The preceding components may exchange signals with each other via a controller bus. In addition, the optical source 210, detector source 211, filter 212, display 214 and an ultrasound generator 207 may exchange signals with the controller 215 via the system bus 216.

The controller 215 typically operates the optical source, 202, ultrasound generator 207, optical detector 210, detector source 211 detector, filter 212 and display 214 through the I/O circuits in response to data and program code instructions stored and retrieved by the memory and executed by the processor. The program code instructions may implement embodiments of the diagnostic technique described herein. The code may conform to anyone of a number of different programming languages such as Assembly, C++, JAVA, Embedded Linux, or a number of other languages. The CPU forms a general-purpose computer that becomes a specific purpose computer when executing program code. Although the program code is described herein as being implemented in software and executed upon a general purpose computer, those skilled in the art will realize that the method of pulsed pumping could alternatively be implemented using hardware such as an application specific integrated circuit (ASIC) or FPGA or other hardware circuitry. As such, it should be understood that embodiments of the disclosure can be implemented, in whole or in part, in software, hardware or some combination of both.

Operation of the apparatus 200 may be understood with respect to the close-up schematic diagram depicted in FIG. 2B. An embedded target structure within the body 201 such as an artery AR or vein VE can be identified by ultrasound imaging.

The ultrasound generator 207 and transducer 206 can be used to do both the ultrasound imaging and the target modulation. Once a target has been located, the apparatus 200 switches between a regular ultrasound mode (imaging) and a radiation pressure modulation mode, firing tone bursts to modulate the target. The basic approach is first to image to choose a location to deliver radiation pressure and then to apply the appropriate phase to the array elements of the transducer 206 to have a focus at the location of interest. The radiation pressure is supplied by applying a tone burst (many cycles of electrical signal at the frequency of operation of the array) from the ultrasound generator 207 to the elements of the array in the transducer 206. The repetition rate at which the tone burst is applied is the frequency at which the radiation pressure is applied. This repetition rate is constrained at the upper end by the fundamental frequency of the ultrasound transducer 206, i.e., the tone burst cannot have a higher repetition rate than the fundamental frequency of the transducer itself. By way of example, the ultrasound transducer 206 can operate at fundamental frequencies in the range 2-50 MHz, and preferably from 100 KHz-50 MHz. The tone bursts may produce radiation pressure modulation occurring at the pulse repetition frequencies between 50 Hz and 750 kHz.

The sensor 203 is then placed proximate to a tissue boundary TB of the boundary 201. The target structure is then vibrated using radiation pressure from the transducer 206 and illuminated with a diffuse photon field with a characteristic frequency ω_{INJ} delivered from the optical source 202 via the launch optics 204. The radiation-pressure modulation of the target is detected by its effect on the emerging photon field at the detector (e.g., via the collecting optics 208). In the example depicted in FIGS. 1 and 2A, it may be possible to measure both venous and arterial oxygenation separately by illuminating and modulating the vein and then separately illuminating and modulating the artery. In the case where the target is the internal jugular vein, the corresponding arterial structure is the carotid artery. This method, when it can be used, will implicitly provide a calibration signal. Cardiac output can then be calculated from the Fick Principle, as described above.

To make the measurement a biological structure within the body 201, such as the pulmonary artery, descending branch of the aorta, internal jugular, or external jugular, is located in a standard manner with medical imaging. Once found, the combined ultrasound/optical sensor 203 can be positioned proximate to the targeted structure. This can either be external dermal placement, e.g., on the neck in the case of the internal jugular vein, or an inserted catheter, either endotracheally for direct access to the left pulmonary artery and thoracic aorta, or trans-esophageally for access to the right pulmonary artery. The sensor 203 is preferably positioned such that the distance between the emitting tip of the launch optics 204 and the lumen of the targeted vessel is approximately minimized.

The ultrasound transducer 206 is used to physically modulate (vibrate) the selected target using ultrasound radiation pressure. The ultrasound transducer 206 is designed to focus its acoustic output into the target at various modulation frequencies. Examples of ultrasound transceivers that can provide such focused output include phased array ultrasound transceivers and single element ultrasound transducers with imaging designs. Phased array transducers typically have an array of ultrasound transducer elements that are narrow and have a wide acceptance angle so that energy from various angles is collected, and so that several elements (if not all) in the array contribute to the focusing at a certain location. To generate a beam, the various transducer elements are pulsed at slightly different times. By precisely controlling the delays between the transducer elements, beams of various angles, focal distance, and focal spot size can be produced. Furthermore, for a given point within the targeted tissue a unique set of delays will maximize the constructive interference of acoustic signals from each of the transducer elements. Such transducers can therefore selectively modulate particular structures within the target without modulating surrounding structures. Beam forming in ultrasound refers to the use of signal processing in order to focus the energy from various transducer elements. The energy is preferentially deposited using focusing to allow the application of radiation pressure at the location of interest with a relatively low level of input signal.

Examples of suitable ultrasound transducers include, for example, the GE Logiq 7 made by General Electric of Fairfield, Conn., or the Aspen® Echocardiography System made by Siemens (Acuson) of Mountain View, Calif. Other suitable array transducers are made by Philips (The Netherlands), or Hitachi (Japan). It is best to choose an instrument that is used commonly in hospitals say to image the heart.

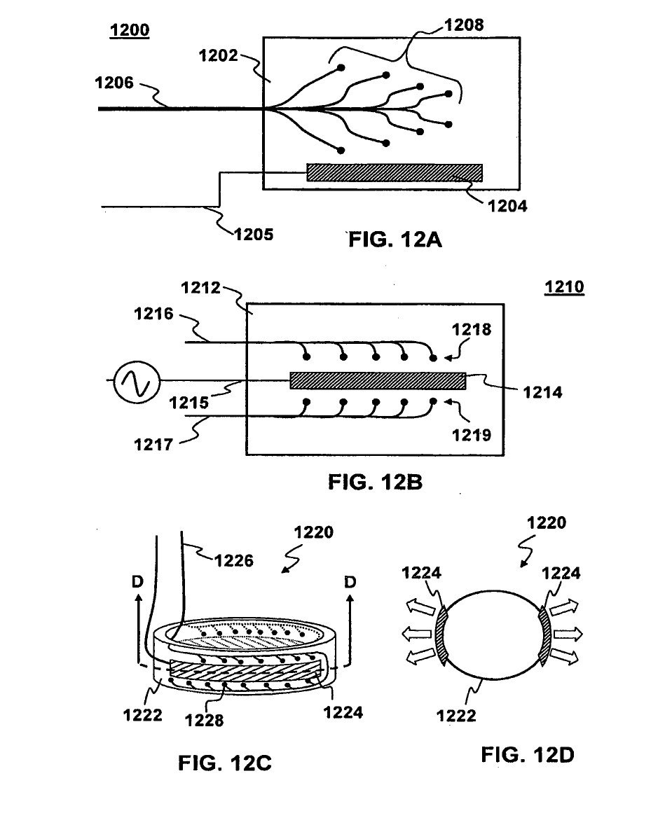

An ultrasound imaging system can also be used in association with the ultrasound generator 207 and transducer 206 to locate the blood vessels in order to orient the delivery of the pulsed or continuous radiation from the optical source 202. The imaging system can be incorporated into the system controller 215. The transducer 206 can be a piezo type transducer as used in the above-described commercially-available ultrasound machines or a cMUT (capacitative Micromachined Ultrasonic Transducer), see X. Jin, I. Ladabaum, B. T. Khuri-Yakub, “The Microfabrication of Capacitive Ultrasonic Transducers,” J. Microelectromechanical Systems, vol. 7, pp. 295-302, September 1998, and U.S. Pat. No. 6,262,946 to Khuri-Yakub et al., both of which are incorporated herein by reference. Using the cMUT will allow a very compact 2-D array to be made. Such compact arrays are very important for ring-shaped transducers such as that shown in FIGS. 12C-12D for the trans-tracheal/trans-esophageal applications.

Using an array-type ultrasonic transducer, one can focus the ultrasound energy on a target structure such as a vein or an artery. While focusing on the vein or artery, the oxygenation level can be measured by modulating the optical energy that is scattered from within the vein or artery, preferably allowing for a direct calibration of the optical signal. For example one can steer the beam from internal jugular to carotid artery, alternatively sampling 100% oxygen saturated blood and the venous blood with reduced saturation. The ultrasound imaging system can also be used to derive the width of the arteries and veins, and the blood flow velocity using Doppler shift of the scattered ultrasound. Such a measurement can provide an estimate of the cardiac output that can be compared to cardiac output as derived from the use of the apparatus 200. This adjunct measurement will have additional diagnostic value as discussed above for the diagnosis of shunts, septic and cardiogenic shock, etc.

Once the ultrasound transducer 206 and launch optics 204 are aligned with respect to the targeted vessels, the array of transducers in the ultrasound imaging system will all be fired, with appropriate phase delays, with a burst of energy to deliver radiation pressure at the focus as determined by the phase delays. The focus of the acoustic signal can be chosen to be inside the vessel acting on the blood cells, or on the side walls of arteries. The radiation pressure associated with the acoustic pulse which is equal to the acoustic intensity divided by the speed of sound in the medium, will act to impart a movement on the cells or arterial walls on which it acts. The use of radiation pressure (alternatively “radiation force”) to induce motion in a target which is then detected by conventional ultrasound techniques has been described by Nightingale et al., “Acoustic Radiation Force Impulse Imaging: In Vivo Demonstration of Clinical Feasibility,” Ultrasound in Medicine and Biology, 28(2): 227-235, (2002) and in U.S. Pat. No. 6,371,912 to Nightingale et al., both of which are incorporated herein by reference. Embodiments of the present disclosure are superior to this technique in that they will permit functional (oxygenation) information to be derived from the target, whereas in the aforementioned prior art, only mechanical information (stiffness, elasticity etc) is derived.

In this fashion, the optical signal, which relates to the oxygen content in the blood cells in the target volume, will be modulated at the frequency at which the radiation pressure pulse is applied, ω_{RPM}. For instance, using a 7.5 MHz imaging system, one can use a burst of, say, 10 cycles at any repetition rate up to around 750 kHz as determined by the physical and mechanical properties of the target and the experimental implementation. It may be possible to tune the interpulse spacing (the repetition rate) in the tone burst to resonantly modulate the target depending on its elastic properties. It may also be possible to tune the ultrasound fundamental frequency to optimize its interaction with the desired target (blood cells, vessel walls etc). In this manner, the detector 210 may detect only those photons which have interacted with the desired target 201.

The optical source 202 may be configured to deliver the temporally correlated groups of photons at a repetition rate of between about 100 kHz and about 500 MHz, preferably between about 1 MHz and about 250 MHZ, more preferably between about 10 MHz and about 200 MHz. The groups of photons may be in the form of pulses having pulse widths in the range of about 1 picosecond to about 1 nanosecond, preferably, about 1 to 100 picoseconds, more preferably about 5 to 50 picoseconds. Alternatively, optical source 202 is configured to deliver continuous wave radiation. The photons may be characterized by wavelengths between about 650 nm and about 1175 nm, preferably between about 650 nm and about 930 nm or between about 1020 nm and about 1150 nm.

The optical source 202 provides temporally correlated photons or continuous wave radiation at two or more different wavelengths. For example radiation from a pulsed or continuous wave laser may be incident on a device that converts radiation at the fundamental frequency of the laser into a pair of photons at two different predetermined frequencies. Such a device could be a nonlinear crystal causing Spontaneous Parametric Down Conversion (SPDC) as, for example, described by Shi and Tomita, “Highly efficient generation of pulsed photon pairs with bulk periodically poled potassium titanyl phosphate,” J. Opt. Soc. Am. B., 21(12) 2081-2084 (2004), or a highly non-linear fiber source as described by Rarity et al., “Photonic crystal fiber source of correlated photon pairs,” Opt. Exp., 13(2), 534-544 (2005).

Alternatively, the optical source 202 may include a non-linear crystal phased matched to act as an optical parametric oscillator (OPO) to provide a temporally correlated photon pair. An OPO takes a fundamental electromagnetic wave at frequency ω_{P1} and converts it to two new frequencies called the signal and idler, ω_{SIG} and ω_{IDL} related by the equation

ω_{P1}=ω_{sig}+ω_{idl}

where the signal and idler waves are emitted in temporal coincidence.

The OPO may be driven by the second harmonic of a pulsed laser operating at a fundamental frequency ω_{P1} to create two new frequencies called the signal and idler, ω_{SIG} and ω_{IDL} related by the equation:

2ω_{P1}=ω_{sig}+ω_{idl}

where 2ω_{P1} is the second harmonic of the fundamental frequency. For example the drive laser may be a mode-locked or Q-switched Nd:YAG laser operating at 1064 nm, giving a second harmonic wave at 532 nm. This wave in turn is used to drive the OPO. In this manner three clinically useful, temporally coincident photon waves at 1064 (ω_{P1}), 1030 (ω_{sig}) and 1100 (ω_{idl}) may be generated. The nonlinear crystal may be selected from a variety of substances, for example BBO, LBO, KTP, KTA, RTP, or periodically poled materials such as periodically poled lithium Niobate (PPLN), periodically poled stoichiometric lithium tantalate (PP-SLT) and the like. Such materials are described, e.g., in the freeware program SNLO distributed by Sandia National Laboratories, Albuquerque, N. Mex.

By way of example, the optical source 202 may include a pulsed solid state laser, for example a picosecond mode-locked laser such as the picoTRAIN™ series compact, all-diode-pumped, solid state picosecond oscillator manufactured by High-Q Lasers of Kaiser-Franz-Josef-Str. 61 A-6845 Hohenems, Austria. The source may also be a mode-locked fiber laser, such as the picosecond version of the Femtolite™ D-200 from IMRA America Inc., Ann Arbor Mich. 48105. Alternatively, a picosecond pulsed diode such as the PicoTA amplified picosecond pulsed laser diode heads manufactured by Picoquant, of Berlin, Germany, may be used as the optical source 202. Note that in the case where continuous wave light is to be used, these mechanisms are not necessary. The optical fibers 205 coupling the optical source 202 to the launch optics 204 may be, e.g., single mode fiber optic, such as the P1-980A-FC-2—Single Mode Fiber Patch Cable, 2 m, FC/PC manufactured by Thorlabs, Inc. of Newton, N.J. Radiation coupled from the optical source 202 to the target 201 via the launch optics 204 is used, e.g., to illuminate the lumen of a selected blood vessel with pulses of or continuous radiation at two or more different wavelengths carefully chosen to have deep penetration into tissue, to have differing affinities for oxy- and deoxy-hemoglobin, or for oxy-hemoglobin and met-hemoglobin, but to have substantially similar scattering cross-sections and anisotropy parameters.

Some of the radiation scatters from the target 201 and is collected by the collecting optics 208 and/or detector 210. By detecting pairs or multiplets of photons at different wavelengths returning from the target tissue in substantial temporal coincidence, it can be inferred that the coincident photons have traveled approximately the same path length in the tissue. This is the main difference between making measurements in clear transparent media where the Beer-Lambert law may be presumed to apply, and making measurements in turbid media where elastic scattering causes a substantial and generally indeterminate pathlength increase, as discussed by Okui and Okada, “Wavelength dependence of cross-talk in dual-wavelength measurement of oxy- and deoxy-hemoglobin,” J. Biomed. Opt., 19(1), 011015 (2005).

The detector is coupled to a filter 212 that selects coincident photon signals having modulation at the radiation pressure modulation frequency or a harmonic thereof. The filter 212 may be coupled to the display 214, e.g., a CRT screen, flat panel screen, computer monitor, or the like, that displays the results of the aforementioned process in a manner readily interpretable, e.g., in the form of text, numerals, graphical symbols or images.

By detecting arrival rates of pairs or multiplets of photons at the frequency of the radiation pressure modulation or a harmonic of the radiation pressure modulation frequency, one can infer that these photons interacted with the radiation-pressure-modulated target 201. If the target 201 contains the oxygenated or deoxygenated forms of hemoglobin (Hb), the detected pair or multiplet coincidence rate will be altered depending on how the wavelengths were selected. The extent to which the detection rate is altered can be correlated to the oxygenation level of the target or to the pH in the target. The met-hemoglobin absorption spectrum is dependent on pH as shown in Zijlstra et al., “Visible and Near Infrared Absorption Spectra of Human and Animal Haemoglobin,” 1^{st} ed. Utrecht: VSP Publishing, 2000, page 62. Thus a non-invasive probe of met-Hb absorption may be used to probe the pH of the structure being targeted.

There are many possible configurations for the optical source 202 of FIG. 2A. For example, FIG. 3 is a schematic diagram of a three-wavelength pulsed optical source 300 that emits three laser pulses at the three wavelengths with temporal coincidence. This could be the OPO source described above. Alternatively, the source 300 generally includes a pulsed laser 302, a seed source 304, and a highly non-linear fiber (HNLF) 306. According to some embodiments, a continuous laser source is used instead of pulsed laser 302. Optics 308, such as one or more lenses, couple pump radiation at a drive frequency ωₚ to the HNLF 306. A 2×2 coupler 310 couples seed radiation at a frequency ωₛ from the seed source 304 into the HNLF 306. When ωₚ and ωₛ are properly chosen, the HNLF 306 acts as an optical parametric amplifier (OPA) that produces three temporally correlated electromagnetic waves at three frequencies: pump radiation at ωₚ, amplified seed radiation at ∫ₛ and idler radiation at an idler frequency ω_{idl} given by:

ω_{idl}=2ωₚ−ωₛ.

For example, if ωₚ corresponds to a vacuum wavelength of 1064 nm and ωₛ corresponds to a vacuum wavelength of 1100 nm, ω_{idl} corresponds to a vacuum wavelength of about 1030 nm.

The fiber 306 preferably has a non-linearity that is high enough to allow non-linear optical effects to occur efficiently in a reasonable length of fiber, and where the non-linearity is sufficiently fast to create the temporal synchronization between the pump, seed and idler waves. Such fibers may be obtained from Crystal Fibre of Birkenrød, Denmark, for example the NL-5.0-1065 type. The non-linear optics underlying the conversion have been described by for example, Ho et al., “Narrow-linewidth idler generation in fiber four-wave mixing and parametric amplification by dithering two pumps in opposition of phase,” J. Lightwave. Tech., 20(3), 469-476 (2002), which is incorporated herein by reference. The drive frequency ωₚ may be provided by a high repetition rate mode-locked picosecond laser, such as the picoTRAIN™ series compact, all-diode-pumped, solid state picosecond oscillator manufactured by High-Q lasers of Kaiser-Franz-Josef-Str. 61 A-6845 Hohenems, Austria or a mode-locked fiber laser, such as the picosecond version of the Femtolite™ D-200 from IMRA America Inc., Ann Arbor, Mich. 48105.

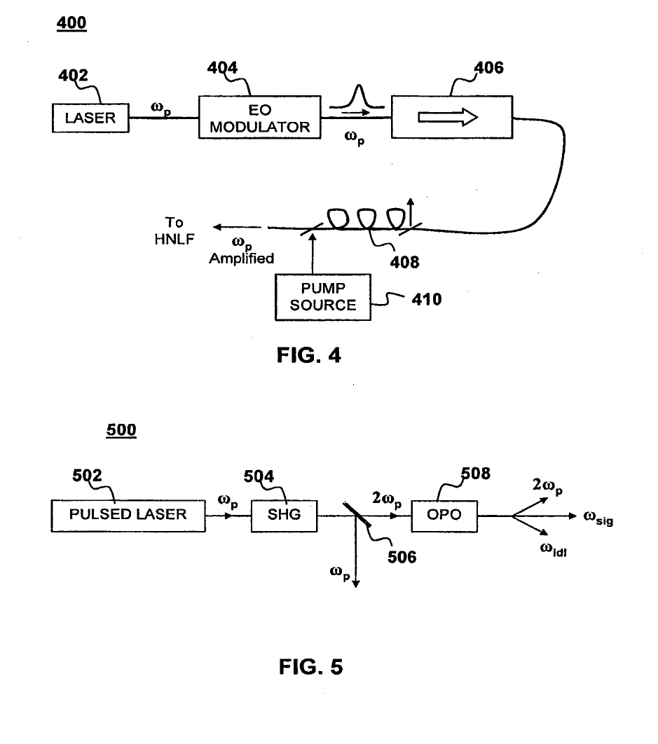

In the source 300, the seed source 304 may be a distributed feedback (DFB) or DBR (Distributed Bragg Reflector) laser, for example the EYP-DBR-1063-00100-2000-S0T02-0000 diode laser manufactured by Eagleyard Photonics, Berlin Germany. There are a number of different possible configurations for the pulsed laser 302. Generally, the pulsed laser 302 should be capable of providing picosecond pulses of pump radiation to the HNLF 306. FIG. 4 is a schematic diagram of an all-electronic optical source 400 of picosecond pulses which could be used as the pulsed laser 302 of FIG. 3. The source 400 generally includes a diode laser 402 an electro-optic modulator (EO) 404 a Faraday isolator 406 and a doped fiber amplifier 408. The diode laser 402 provides radiation at ω_{P} which is modulated by the EO modulator 404 to create weak picosecond radiation pulses 401, which are coupled to the fiber amplifier 408. The Faraday isolator 406 transmits pulses to the fiber amplifier 408 and blocks radiation from being reflected back towards the EO modulator. A fiber pump source 410 provides fiber pump radiation (e.g., at a vacuum wavelength of 980 nm) to the cladding or core of the fiber amplifier 408. The fiber amplifier may include a dump for the pump laser so that fiber pump radiation does not oscillate through fiber amplifier 408. The amplifier 408 amplifies the weak pulses 401 to create amplified pulses 403 that can be fed to the HNLF 306.

By way of example, the diode laser 402 is a continuous wave (CW) tunable DFB or DBR diode laser, such as the EYP-DBR-1063-00100-2000-S0T02-0000 diode laser manufactured by Eagleyard Photonics, Berlin Germany The EO modulator 404 may be a Model 4853 6.8/9.2-GHz Modulator from New Focus (Bookham) San Jose, Calif. The Faraday isolator 406 may be a model 4I1055 from Electro-Optic technology, of Traverse City, Mich. The fiber amplifier 408 may be doped with Ytterbium or Neodymium, such as the DC-225-22-Yb made by Crystal Fibre (Birkerod, Denmark). The fiber pump may for example be a model 4800, 4 W, Uncooled, Multi-Mode pump module from JDS Uniphase, of San Jose, Calif.

According to some embodiments, where continuous laser light is used instead of pulsed laser energy, EO modulator 404 and fiber amplifier 408 can be omitted.

As discussed above, the optical source 202 may produce the correlated photons by optical parametric oscillation. FIG. 5 is an example of such an optical source 500. The source 500 generally includes a pulsed laser 502, a second harmonic generator (SHG) 504, a dichroic mirror 506 and an optical parametric oscillator (OPO) 508. The pulsed laser produces pump radiation at a frequency ωₚ. The second harmonic generator interacts with the pump radiation to produce second harmonic radiation at double the frequency of the pump radiation, i.e., at 2ωₚ. The SHG 504 may be less than 100% efficient at doubling the frequency of the pump radiation. The dichroic mirror 506 deflects pump radiation that makes it through the SHG 504. In the OPO 508, some of the second harmonic radiation is converted to signal and idler radiation, respectively at frequencies ω_{sig} and ω_{idl} that are related by:

2ωₚ=ω_{sig}−ω_{idl}

The pulsed laser 502 may be of any of the types described above. The second harmonic generator may be a non-linear crystal of any of the types described above phased matched for second harmonic generation. The OPO 508 may be a non-linear crystal of any of the types described above phased matched for optical parametric oscillation. The source 500 has the advantage of being tunable by virtue of the OPO phase matching. The phase matching is typically tuned by adjusting e.g., the angle of the non-linear crystal used in the OPO, or by changing its temperature. Alternatively the poling period may be adjusted in periodically poled materials to phase match at different wavelengths.

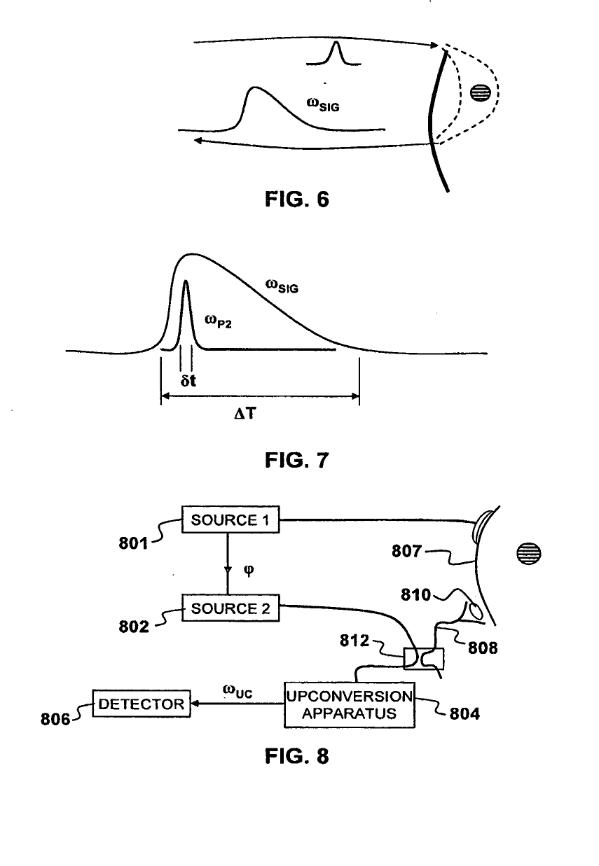

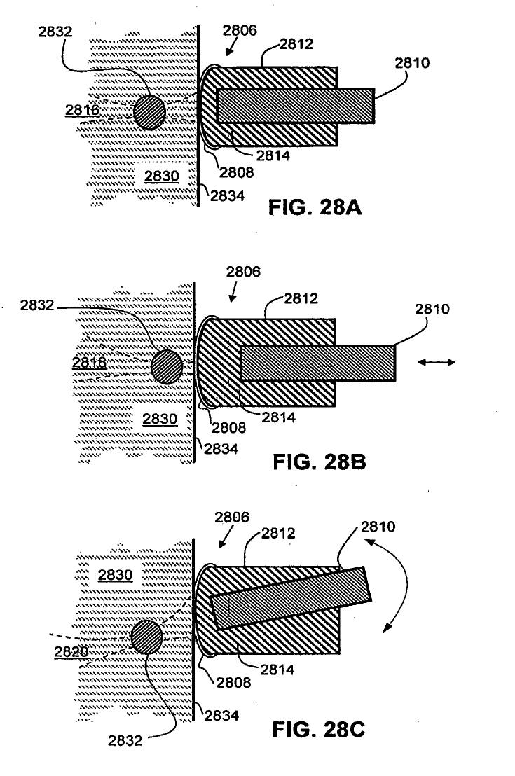

Radiation pulses from the source 200 may be affected by traveling through tissue. For example, FIG. 6 is a schematic diagram of the signal expected at the tissue boundary TB shown in FIG. 28. Injected pulses of radiation at frequency ωᵢₙⱼ with short pulse widths (e.g., about 1 to 50 picoseconds) are delivered into the body 201 at the tissue boundary TB. An injected pulse interacts with tissues in the body and emerges as a signal pulse at an optical frequency ω_{sig}, which may be slightly different from ωᵢₙⱼ as a result of interaction with the ultrasound pulse. However, any frequency shift occurring as a result of interaction between the optical pulses and the ultrasound pulses will be insignificant compared to the natural linewidth of the picosecond laser pulse as a result of the time-bandwidth constraint which derives directly from the Heisenberg Uncertainty Principle. Furthermore, detection of this ultrasound-induced frequency shift is not required in the proposed embodiments of the disclosure, distinguishing this technique from those in the prior art. The signal pulse is typically broadened (e.g., to a pulse width of several hundred picoseconds to several nanoseconds) compared to the injected pulse due to the random-walk nature of photon propagation in turbid media, as shown by Turner et al., “Complete-angle projection diffuse optical tomography by use of early photons,” Opt. Lett., 30(4), 409-411 (2005). This random walk increases the effective path length considerably. The time at which the photon arrives at the tissue boundary may be related to its approximate path length through mathematical relationships, for example the diffusion approximation or the Transport Equation.

The pulse spreading described above is taken into account in time-gated detection of the signal pulse. One possible approach to taking such pulse spreading into account utilizes a technique referred to herein as time gated upconversion. FIG. 7 is a schematic diagram illustrating the principle of time gated upconversion. The broadened signal pulse at ω_{sig} emerging from the tissue boundary TB with a pulse width ΔT of, e.g., a few nanoseconds, is mixed with a short mixing pulse (e.g., pulse width δt of order several picoseconds) of radiation at an optical frequency ω_{P2}. A master oscillator or a secondary slave oscillator may provide the short mixing pulse at ω_{P2}. The mixing takes place in an upconverter such as a fiber OPA or a mixing crystal. Mixing can only occur when the two pulses are temporally and physically overlapped, so by strobing the mixing pulse through the emerging signal pulse, it is possible to time gate the signal that is to be detected. This upconversion process may be accomplished in a manner that is highly efficient as described by Langrock et al., “Sum-frequency generation in a PPLN waveguide for efficient single-photon detection at communication wavelengths,” Stanford Photonics Research Center Annual Report, (2003) D-19-D-21, which is incorporated herein by reference.

FIG. 8 is a schematic diagram illustrating of an alternative optical signal generation and detection apparatus 800 for use with embodiments of the present disclosure. The apparatus 800 includes first and second pulsed optical sources 801, 802 that respectively produce pulsed optical signals at optical frequencies ω_{P1} and ω_{P2}. The first source 801 serves as a master oscillator for timing purposes and its output is used in one of the aforementioned processes to create two or more pulses of light at two or more wavelengths selected per the criteria described above. A timing signal φ is derived from the first source 801 and used to trigger the second source 802, which operates at substantially the same pulse repetition rate as the first source 801, but with an adjustable delay (phase angle) between the two pulse trains. The pulse train from the second source 802 is mixed in an upconversion apparatus 804 with the emerging signal at optical frequency ω_{SIG} from a tissue boundary 807 and the time delay between the two sources is adjusted to temporally gate the resulting signal, which is detected at a detector 806. This permits background-free, time-gated analysis of the emerging signal. The resulting upconverted signal may have an optical frequency ω_{UC} of ω_{P2}+ω_{SIG} or 2ω_{P2}−ω_{SIG} depending on the nature of the upconversion apparatus 804. The two signals may be mixed, e.g., using a relay fiber 808 coupled to collection optics 810 and a 2×2 coupler 812 coupled ‘to the relay optics and the second source 802.

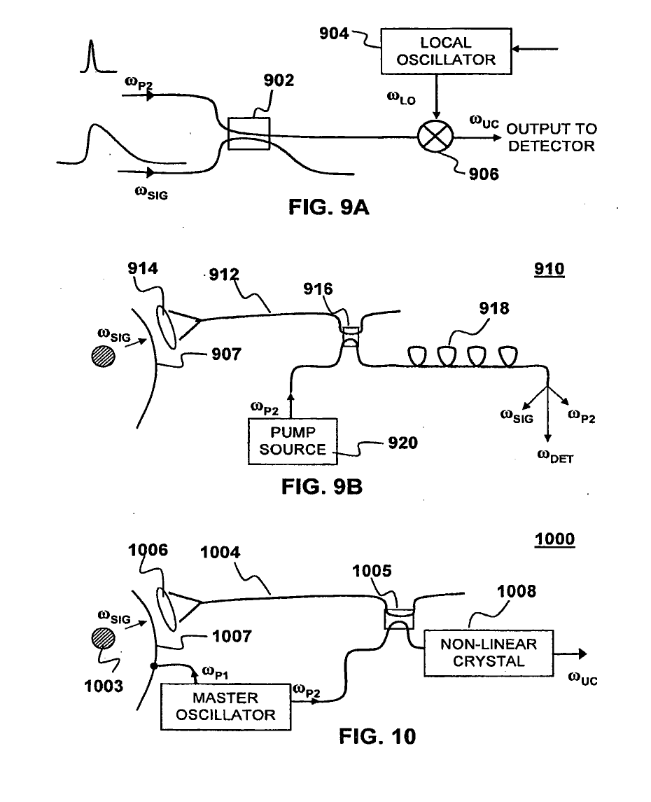

In some embodiments, the upconversion apparatus 804 may include a local oscillator, e.g., a laser for time-gated upconversion. For example, as depicted in FIG. 9A, the signal pulse at ω_{SIG} and mixing pulse at ω_{P2} are combined, e.g., using a 2×2 coupler 902. Upconversion as described above may then be used to create a new signal photon wave at either ω_{P2}+ω_{SIG} or 2ω_{P2}−ω_{SIG}. A local oscillator laser 904 produces a pulsed wave at an optical frequency ω_{LO} and a repetition rate correlated to the ultrasound tone burst that is mixed in a mixing stage 906 with the new signal pulses before detection, generating a composite wave at optical frequency ω_{UC} given by either (ω_{P2}+ω_{SIG}+ω_{LO}) or (2ω_{P2}−ω_{SIG}+ω_{LO}) that is coupled to the detector 210. The mixing stage 906 may be a waveguide of for example a PPLN or PP-SLT, or a crystal of KTP or other material with high optical non-linearity. In this manner, a signal may be generated that is temporally selected for an effective path length in the tissue. Upconverting the signal from the near-IR (around 1 micron) to the visible (400-700 nm) in this manner allows the use of silicon-based detector technology that has several advantages over InGaAs technology as discussed by Langrock et al. For example benefits include greater receiver sensitivity and lower dark counts from the detector.

The signal may be further selected for a temporal relationship to the modulating ultrasound tone burst from the transducer 206 by triggering the local oscillator 904 with an appropriate reference signal from the ultrasound source 207. For example, by triggering the local oscillator 904 at twice the repetition rate of the tone burst, one can make a direct on/off comparison between the signal coming back from the tissue in the presence of, and absent the effect of the mechanical modulation.

Alternatively, the upconversion apparatus 804 may provide background free time gated amplification of the signal pulse. This may alternatively be accomplished using fiber Optical Parametric Amplification, e.g., as depicted in FIG. 9B. In an OPA-based background-free time-gated upconversion detector 910, optical signals at optical frequency ω_{SIG} emerging at a tissue boundary 907 are coupled into a relay fiber 912 by collection optics 914. The emerging optical signals at ω_{SIG} are then mixed (e.g., using a 2×2 coupler 916) into a Highly Non-Linear Fiber (HNLF) 918 with a drive pulse at optical frequency ω_{P2} from a pump source 920. The drive frequency ω_{P} may be provided by a high repetition rate mode-locked picosecond laser, such as the picoTRAINT™ series compact, all-diode-pumped, solid state picosecond oscillator manufactured by High-Q lasers of Kaiser-Franz-Josef-Str. 61 A-6845 Hohenems, Austria or a mode-locked fiber laser, such as the picosecond version of the Femtolite™ D-200 from IMRA America Inc., Ann Arbor, Mich. 48105. The signal at ω_{SIG} is converted to a detected signal at ω_{DET} by an Optical Parametric Amplification (OPA) process in the fiber 918. The OPA process creates the detectable signal ω_{DET}, e.g., through a four-wave mixing process described by:

ω_{DET}=2ω_{P2}−ω_{SIG}

Since the upconversion process only happens when the drive pulse at ω_{P2} is present the upconversion can be time gated. It should be noted that the frequency W_{DET} of the detected signal is higher than either the signal or drive frequencies respectively. This means that the signal detected at frequency ω_{DET} will be substantially free of contaminating signals, e.g., from tissue autofluorescence (which always occurs to longer wavelength than the excitation wavelength), inelastic scattering internal to the fiber (Raman scattering for example) which is also always to longer wavelength than the fundamental, and other non-linear inelastic processes. By delaying the onset of the mixing or upconversion pulse used in the detection stage (802, 920), and then lengthening it in time using for example the all-electronic source shown in FIG. 4, we may adjust the detector to:

a) eliminate signal from photons which could not have interacted with the target, and

b) include all possible contributions from photons which could have interacted with the target. This is equivalent to applying a Heaviside (step) function to the detected signal.

The aforementioned detection method may be more efficient compared to slowly moving a short upconversion/mixing pulse through the temporally broadened signal (FIGS. 6 and 7) by varying the delay as this latter technique implicitly selects a small subset of the photon trajectories, ignoring other possible contributions.

The detected signal may be amplified in a time gated manner by selecting a delay between the signal at ω_{P1} from the tissue boundary to be amplified and the drive pulse at ω_{P2}. The drive pulse may be part of the beam from a master laser or may preferably be produced by a second pulsed laser operating at similar repetition rate and pulsewidth to the master oscillator. The amplification of a particular segment of the returning signal may also be selected by overlapping the two signals in time using a variable delay line. Using this technique, the signal at ω_{P1} will also be amplified by gains of for example 10-60 dB, as described by Ho et al. and references therein, allowing very weak signals to be detected.

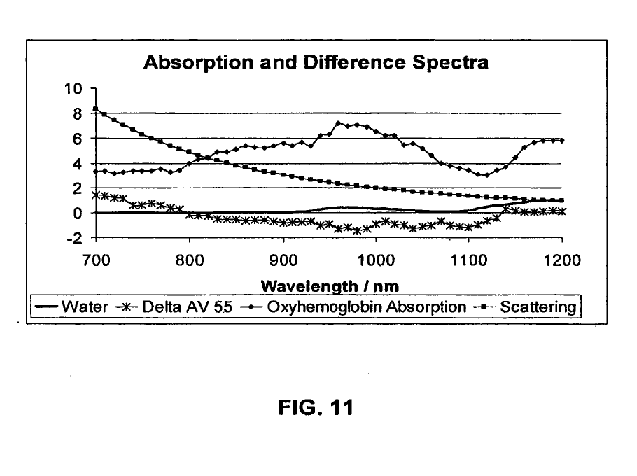

Other background-free time-gated upconversion detection schemes can be implemented. For example, FIG. 10 depicts an alternative background-free time-gated upconversion detector 1000. In the detector 1000, a master oscillator 1002 produces a first master oscillator pulse at an optical frequency ω_{P1}. The first master oscillator pulse is used to generate temporally correlated photons (e.g., as described above) that are scattered from a target tissue 1003 within a body 1001 to provide a signal. Signal photons at an optical frequency ω_{SIG} emerging from a tissue boundary 1007 are coupled into a fiber 1004, e.g., via relay optics 1006. After amplification in a doped section of the fiber 1004, the signal photons are mixed (e.g., using a 2×2 coupler 1005) in a non-linear crystal 1008 with a second time-delayed master oscillator pulse having an optical frequency ω_{P2}. The non-linear crystal 1008 is phase matched for frequency mixing of the signal photons and the second oscillator pulse. The resulting upconverted signal is characterized by an optical frequency ω_{UC} given by:

ω_{UC}=ω_{P2}+ω_{SIG}.

A temporal delay between the first and second oscillator pulses is adjusted such that the time evolution of the signal emerging from the tissue boundary can be probed. This allows early arrival photons, which could not have interacted with the target by virtue of their arrival time, to be gated out.

It should be understood that the signals referred to above generally include two or more signal photons of different wavelengths that are detected in coincidence. Coincidence detection of the two signal photons can be accomplished by balanced photoreceivers, for example New Focus (Bookham) model 1807 and 1817, San Jose, Calif. The wavelengths of interest can be isolated by interference filters such as the RazorEdge™ and MaxLine™ Laser and Raman filters from Semrock, Inc. of Rochester, N.Y. Alternatively coincident photon pairs or multiplets can be detected using high speed analog and digital electronics, for example time correlated single photon counting equipment such as the SPC-134 from Becker and Hickl GmbH, Berlin, Germany, or boxcar integrators such as the Model SR200 Boxcar from Stanford Research Systems, Sunnyvale, Calif.

The time-gated amplified signal is analyzed to reveal the component being modulated at the radiation-pressure modulation frequency ω_{RPM}. This can be accomplished using lock-in detection using for example a lock-in amplifier (e.g., a Stanford Research Systems SRS Model 844) as the filter 212 in FIG. 2A.

The remaining signal by virtue of the above generation and detection techniques has:

a) Interacted with the target structure being modulated by the radiation pressure field,

b) Been generated by photons at each of the two or more selected wavelengths which traveled approximately the same path length from the launch site, through the target being modulated, and back to the detector.

The two or more wavelengths of the correlated photons provided by the optical source 202 may be selected to have different affinities for the various states of hemoglobin (oxy-Hb, met-Hb, deoxy-Hb). The arrival of correlated photons at the different wavelengths therefore can be interpreted to indicate, for example, the oxygenation level or pH of the blood in the modulated target structure. For example, if one radiation-pressure modulates a blood vessel and its contents, and illuminates the area with two wavelengths of light, one selectively absorbed by oxy-hemoglobin and one substantially less selectively absorbed, the arrival rate of correlated photon pairs will be higher if they traverse a radiation-pressure-modulated vascularized area containing high levels of deoxy-Hb (because one of the pair will be selectively more absorbed in areas of higher oxygen saturation). By way of example 1030-nm radiation is absorbed more strongly by oxy-hemoglobin than 1064-nm radiation. Similarly, 1100-nm is more strongly absorbed by oxy-hemoglobin than 1064-nm radiation. These three wavelengths may be conveniently generated as shown above. They also have the added attraction of having substantially similar elastic scattering coefficients, which will lead to a simplification in calculation of the effective path length each traverses. They also have substantially similar absorption in water, leading to a simplification in assessing the potential contribution for error in the measurement caused by non-hemoglobin related absorption of the probe wavelengths.

FIG. 11 is a graph showing the absorption of oxyhemoglobin (diamonds) and water (solid curve) in the range 700-1200 nm, the nominal variation of the scattering coefficient as a function of wavelength (squares), and the expected difference in absorption between an artery with fully oxygen-saturated blood (SaO₂=100) and a representative vein where the oxygen saturation is 55% (asterisks—Delta AV 55). The points at which the difference curve crosses Y=0 are known as isosbestic points. There are two isosbestic points in the absorption spectra of oxy-hemoglobin and deoxy-hemoglobin, one around 810 nm and one around 1135 nm. At these wavelengths the absorption of blood in the vessel is independent of oxygen saturation. These points are known to be useful for internal reference calibration, for example to exclude the effects of volume changes in the absorption resulting from pulsatile flow from the heart.

The wavelength range 1025-1135 nm is characterized by having reduced absorption as the venous oxygen saturation decreases. This means that the signal derived as described in the embodiments of the present disclosure will increase with decreasing saturation in this wavelength range. The gradient of the absorption change with respect to oxygen saturation at the 1135 nm isosbestic point is also very steep, much more so than at 810 nm, making it of significant potential value. Around this wavelength range, we may make sensitive measurements at two or more wavelengths on each side of the isosbestic point. The sign of the absorption change will change from one side of the isosbestic point to the other.

The scattering function in FIG. 11 varies as the inverse fourth power of the wavelength. This means that longer wavelengths (for example from 1025 nm-1150 nm are not as affected by scattering as shorter wavelengths from for example 700-930 nm). This translates to a smaller increase in the effective path length resulting from elastic scattering. The scattering function in the 1025 nm-1150 nm also does not vary significantly, indicating that if we probe the target using wavelengths in this range we may regard scattering as a secondary effect and model it as a perturbation. This is not true in the 700-930 nm range, where the scattering function varies by more than a factor of three.

The wavelength range 1025-1150 nm has rich structure in the difference spectrum, has much lower scattering than the visible and near-IR wavelength ranges, and has relatively modest water absorption. This region offers several convenient and readily available laser sources (Nd:YAG, Yb:Fiber lasers) which are known from dermatology to have excellent penetration properties into tissue.

It is possible to bias the selection of wavelengths to enhance the diagnostic value of the measurement. For example, fetal oxygenation levels are known to be substantially lower than the conjugate maternal levels. Thus, the selection of wavelengths can be biased to probe the fetus preferentially over the mother. Furthermore, if it is desired to detect the pH of the blood in the ultrasound-modulated target, one can inject probe photons at a frequency known to be selective for met-hemoglobin absorption. For example in the wavelength range from 800-1350 nm met-hemoglobin has much stronger absorption than either oxy-hemoglobin or deoxy-hemoglobin as shown in Kuenster J. T. and Norris K. H. “Spectrophotometry of human hemoglobin in the near infrared region from 1000 to 2500 nm,” J. Near Infrared Spectrosc., 259-65 (1994). The wavelength range 1000-1300 nm and especially from 1100-1250 nm is particularly sensitive to met-hemoglobin absorption. The absorption spectrum of met-hemoglobin is known to be sensitive to pH, as shown for example in ZijIstra et al., “Visible and Near Infrared Absorption Spectra of Human and Animal Haemoglobin,” 1^{st} ed. Utrecht: VSP Publishing, 2000, page 62, and one may thus infer the pH of the target from the coincidence arrival rate of appropriately chosen photon pairs or triplets or higher multiplets.

Embodiments of the present disclosure are distinguishable from Diffuse Optical Tomography, where the signal detected has subsumed within it all possible absorbers in the path of the field and no attempt is made to localize the absorber location. The present technique is further distinguished from the various practices of ultrasound-tagged optical spectroscopy because it does not detect small frequency shifts or speckles on the emerging photons. Instead, the present technique detects the modulation imparted by physical motion of the target, which in turn affects the optical absorption cross-section. The present disclosure is insensitive to the very short speckle decorrelation time caused by blood flow in the vessel, which would otherwise severely complicate the detection of modulated photons in interferometric or frequency-domain techniques. The present modulation technique occurs at a much higher frequency than other motion artifacts, for example pulsatile flow from the heart beat, allowing it to be decoupled in the signal analysis. This is important when, for example, the technique is used to perform trans-abdominal fetal oxygenation measurements where it is desirable to distinguish the maternal and fetal oxygenation systems.

There are many possible designs for sensors that may be used in embodiments of the present disclosure. For example, FIG. 12A depicts an example of a sensor 1200 for transdermal measurements. The sensor 1200 generally includes a substrate 1202, which may be made of a flexible plastic or similar material. A thin ultrasound transducer 1204 is mounted on or embedded within the substrate. The transducer 1204 receives power from an ultrasound transmitter and sends return signals through a cable 1205. Optical signals are transmitted and received through an optical fiber bundle 1206 containing launch and receive fibers terminated with coupling optics 1208. The launch/receive fibers and coupling optics 1208 may be mounted to or embedded with the substrate 1202, proximate the transducer 1204. The launch/receive fibers may be used to both transmit and receive optical signals. The fibers and coupling optics 1208 are distributed in a more or less planar fashion. This type of sensor may be used for transdermal measurements.

FIG. 12B depicts an alternative sensor 1210 that is a variation on the sensor shown in FIG. 12A. A transducer 1214, launch fibers and optics 1218, collection fibers and optics 1219 are mounted to or embedded within a substrate 1212 in a more or less planar fashion. In this example, the transducer 1214 is disposed between the launch fibers and the collection fibers. The transducer 1214 receives power from an ultrasound transmitter and sends return signals through a cable 1215. The launch fibers and optics 1218 receive optical radiation from a source via a fiber bundle 1216. The collection fibers and optics 1219 transmit signals to a detector via another fiber bundle 1217.

Other sensor configurations may be useful for trans-esophageal or trans-tracheal measurements. For example, FIGS. 12C-12D depict a sensor 1220 that may be inserted into the esophagus or the trachea. The sensor 1220 includes a ring-shaped substrate 1222 made of a bio-compatible material. Two or more ultrasound transducers (or transducer arrays) 1224 are mounted to the substrate 1222. The transducers are arranged to emit ultrasound in an outward fashion as indicated by the arrows depicted in FIG. 12D. The transducers receive and transmit signals through a cable 1225. Arrays of launch/receive fibers 1228 are disposed on or embedded within the substrate 1222 proximate the transducers 1224. The launch/receive fibers 1228 receive and/or transmit optical signals via a fiber bundle 1226. The ring-shaped sensor 1220 may be placed in the esophagus. Alternatively, the sensor 1220 may be placed in the left or right bronchus, through the trachea, e.g., at the end of a tube that provides oxygen to the patient. Alternatively, the sensor 1220 may be implanted into the patient's trachea and providing a read out to small portable monitoring unit for continuous ambulatory monitoring.

Use of the sensors and apparatus described above for monitoring of blood oxygenation can be accomplished in a variety of different ways.

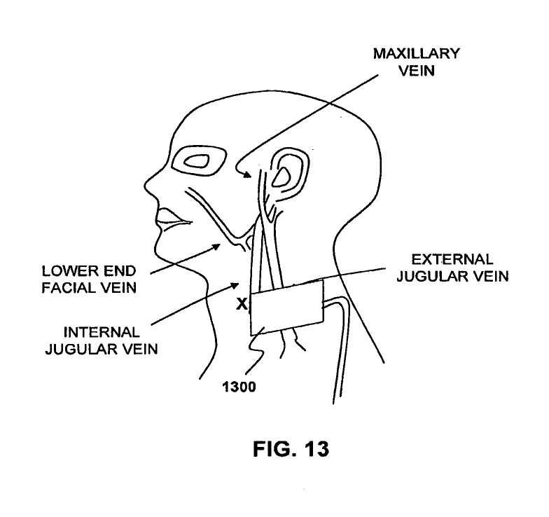

For example, FIG. 13 illustrates a simple case of transdermal measurements of oxygenation in the interior or exterior jugular vein of a patient. A sensor 1300, e.g., of the type depicted in FIG. 12A or FIG. 12B may be placed against the patient's neck in the vicinity of the spot marked with an X. The sensor 1300 may be coupled to a remote unit of the type described above with respect to FIG. 2A. Venous oxygen saturation in the jugular vein can be measured using the ultrasound/optical technique described above while arterial oxygenation can be measured using standard pulse oximetry. Cardiac output can then be calculated from the Fick principle as described above. Alternatively arterial saturation may be measured by radiation-pressure modulating the carotid artery instead of the internal jugular vein. Although a single sensor 1300 is depicted on one side of the of the neck, two or more such sensors (or one large sensor) may be placed on the dermis simultaneously on the left and right side of the neck over both internal jugular veins.

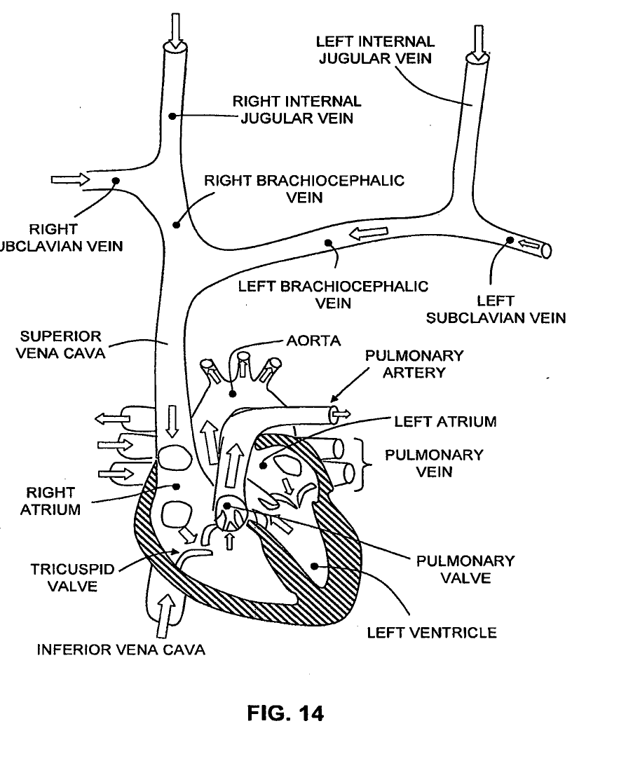

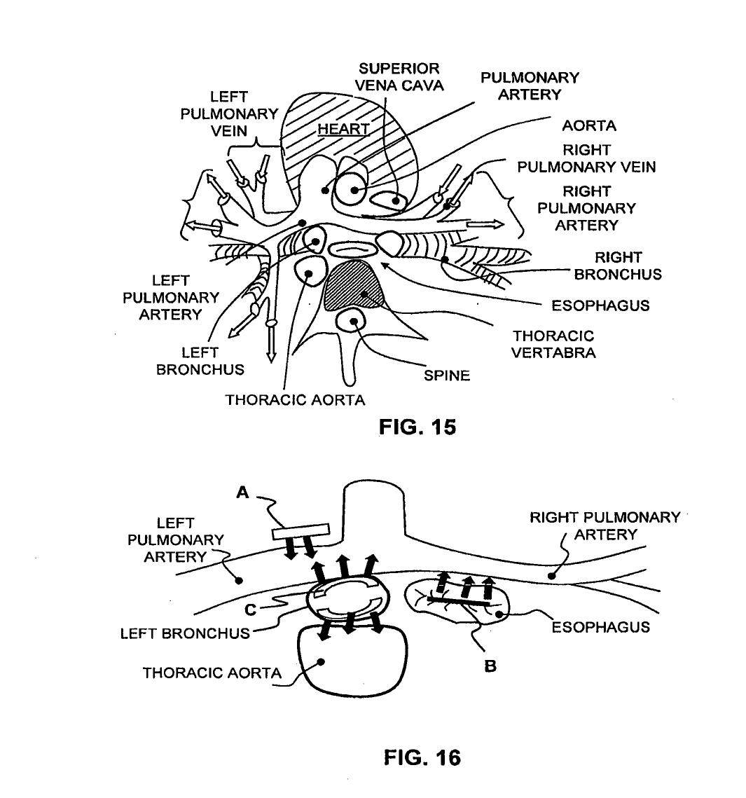

There are a number of different targets within the body that are suitable for blood oxygen monitoring using embodiments of the present disclosure. These can be understood with reference to the anatomical diagrams of FIG. 14 and FIG. 15. For example, both right and left internal jugular veins are potential targets as described above. Measuring both simultaneously would probably be a superior method. FIG. 16 illustrates three other possible sensor placements that may be used in conjunction with embodiments of the present disclosure. First, a sensor A may be inserted using a bronchoscope between two ribs (an intercostal space) next to the sternum. In this case, the sensor could be positioned right up against the pulmonary artery (probably away from the aorta). This is the optimum place to make the measurement of venous oxygen saturation assuming that there are no defects in the heart. For example, if there is an acquired ventricular septal defect, in which blood is short-circuited from left ventricle to right ventricle, the oxygen saturation of the pulmonary artery is abnormally high (e.g., about 80, whereas the incoming blood from the jugular vein may be around 50). Such a condition would result in a false reading for the cardiac output measured using the Fick principle. However, an alternative probe site on the internal jugular vein gives an adjunct measure of the cardiac output independent of heart defects. So the two measurements would be complimentary.

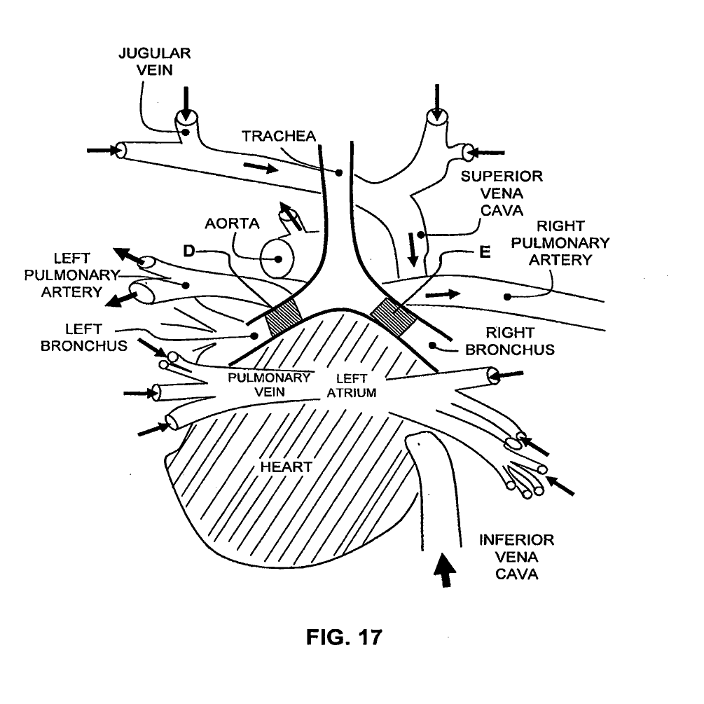

Alternatively, as shown in FIG. 16, a sensor B may be placed in the esophagus. The sensor B may be of the planar type depicted in FIG. 12A or FIG. 12B or the ring type depicted in FIGS. 12C-12D. A sensor C may also be placed in the left bronchus via the trachea. These two probes will also sample the pulmonary arteries. The trans-esophageal probe will sample the right pulmonary artery. The trans-tracheal (bronchial) sensor C will potentially be able to simultaneously probe the oxygen saturation in both the left pulmonary artery (the venous saturation) and the descending thoracic aorta (arterial saturation). This would eliminate the need for external pulse oximetry to measure the arterial oxygen saturation. Positioning of a sensor D within the left bronchus or a sensor E within the right bronchus is illustrated in the dorsal pull-away view of FIG. 17. Such trans-tracheal sensors may be the ring-shaped sensor of the type depicted in FIGS. 12C-12D. The sensors A, B, C, D, E may be coupled to a remote unit of the type described above with respect to FIG. 2A. Optical and ultrasound signals can probe the chemistry of the cardiovascular system in the manner described above.

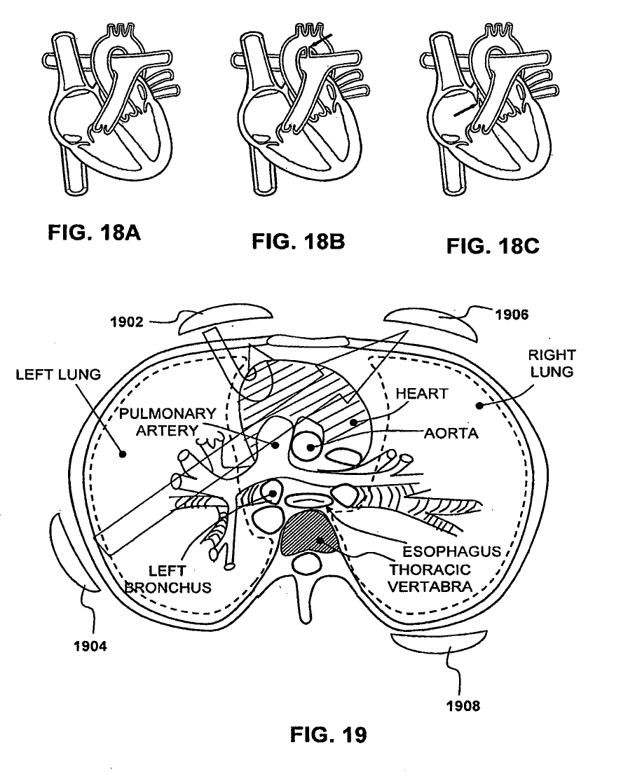

Embodiments of the present disclosure also have application to monitoring of neonatal blood oxygenation. Monitoring of neonatal blood oxygenation is particularly useful in the cases of neonatal heart defects as illustrated in FIGS. 18A-18C. FIG. 18A depicts an example of a normal heart. Certain patients exhibit a heart defect known as Patent Ductus Arteriosus (PDA). As illustrated in FIG. 18B, PDA is the persistence of a normal fetal structure (indicated by the arrow) between the left pulmonary artery and the descending aorta. Persistence of this fetal structure beyond 10 days of life is considered abnormal. Other patients exhibit a defect known as Patent Foramen Ovale (PFO). As shown in FIG. 18C, PFO is a persistent opening in the wall of the heart (indicated by the arrow) which did not close completely after birth. The opening is required before birth for transfer of oxygenated blood via the umbilical cord. This opening can cause a shunt of blood from right to left, but more often there is a movement of blood from the left side of the heart (high pressure) to the right side of the heart (low pressure). Normally this opening closes in the first year of life; however in about 30% of adults a small patent foramen ovale is still present. Diagnosis of both PDA and PFO may be helped by measurement of venous oxygen saturation.

In newborn infants (neonates), the distance across the thorax may be small enough that in addition to trans-esophageal and trans-tracheal, and trans-dermal for the internal jugular, it may be possible to operate the diagnostic apparatus transdermally with a sensor placed directly on a neonate's chest surface. The sensor, e.g., of the planar type depicted in FIGS. 12A-12B, is placed proximate the heart or a blood vessel of interest. The target area is a neonatal cardiovascular system. As illustrated in the cross-sectional diagram of FIG. 19, the measurement may be made in either a reflection mode or trans-illumination mode (in one side—out the other). In the reflection mode, optical signals are transmitted and received via a common sensor 1902. In the trans-illumination mode, a transmitter unit 1904 sends optical signals through an infant's thorax. Scattered photons of radiation from these signals are collected by one or more sensors 1906, 1908 that are positioned to probe radiation scattered from particular structures within the neonatal anatomy such as the pulmonary artery. The sensors 1906, 1908 may be coupled to a remote unit of the type described above with respect to FIG. 2A. Optical and ultrasound signals can probe the chemistry of the neonatal cardiovascular system in the manner described above.

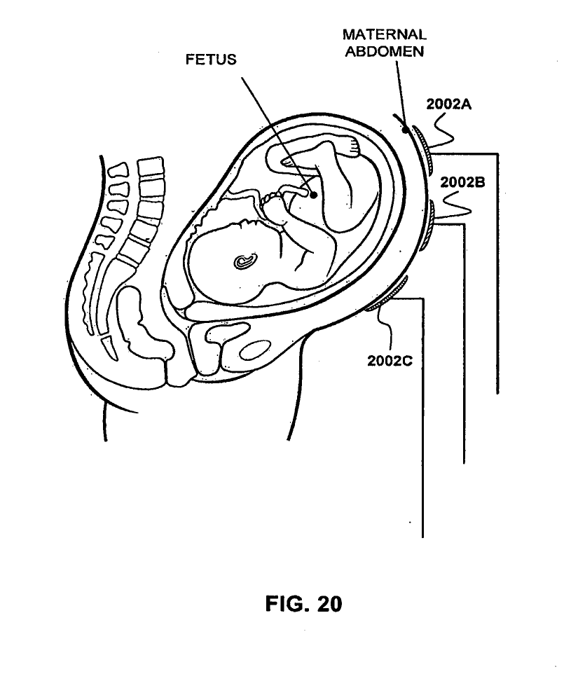

Further embodiments of the disclosure include using diagnostic apparatus of the type described herein for fetal monitoring. For example, as depicted in FIG. 20, one or more sensors 2002A, 2002B, 2002C, e.g., planar sensors of the type depicted in FIGS. 12A-12B, may be placed on a pregnant woman's abdomen to probe the fetal cardiovascular system. The sensors 2002A, 20028, 2002C may be coupled to a remote unit of the type described above with respect to FIG. 2A. Optical and ultrasound signals can probe the chemistry of the fetal cardiovascular system in the manner described above. In this case, the target area is the fetal oxygen exchange system, including the placenta, placental vasculature, fetal heart and major fetal blood vessels. Such trans-abdominal fetal monitoring can provide information about fetal blood oxygenation levels in a minimally invasive or non-invasive manner. Fetal oxygenation levels are known to be substantially lower than the conjugate maternal levels. The selection of wavelengths used can be biased to probe the fetus preferentially over the mother.

Further embodiments of the disclosure will now be described providing greater detail and further examples of systems and methods for inducing changes in blood volume using ultrasonic and other acoustic means.

Conventional ultrasound phased arrays or linear array transducers utilize a ceramic element for each channel. For example, current devices have up to 768 channels. The elements are typically made of a piezoelectric ceramic material. The group of elements used to transmit an ultrasound beam is often referred to as the “transmit aperture.” The transmit signals from the array elements can be individually delayed in time, hence the term “phased array.” This is done to electronically steer and focus each of a sequence of acoustic pulses through the plane or volume in the human body. A larger transmit aperture creates more tightly focused beams concentrating the transmit energy in a smaller target area. Because high transmit pressure waves in the human body can generate cavitations and hence cause harm when the cavitations bubbles collapse, the FDA has setup safety standards for diagnostic ultrasound equipment.

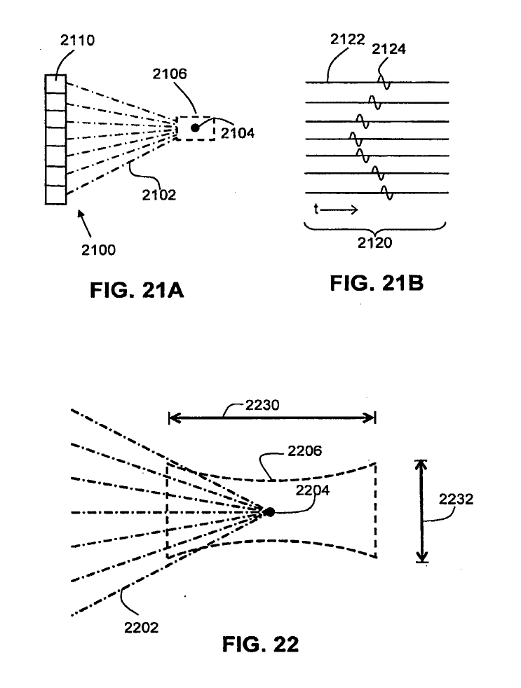

FIGS. 21A-B are a transmit array and an associated transmit time delay profile according to an embodiment of the disclosure. Referring to FIG. 21A, transmit aperture 2100 is made up of a number of individual transducer elements 2110 which, as described above, can be made of piezoelectric ceramic material. Although FIG. 21A only shows 7 elements, in practice, much larger numbers of elements may be used according to the particular application. For example, it is common for transmit arrays to include 128 or 192 transducer elements in a 4 cm total length. In practice, the number of transducer elements of the total array that are used in an application depends on focal length, as well as the desired dimensions of the focal area. For example, if the focal length is 1 cm for a particular target blood vessel, using a 4 cm 192-element transducer array, and an f number of f=1.0 yields an acceptable depth of field, then 1 cm or one-fourth of the 4 cm array should be used (or 48 elements of the 192-element array). When operating transmit aperture 2100 in single beam focusing mode, transmit array 2100 generates a beam of pressure waves at a single focal area 2106. The elements 2110 in transmit aperture 2100 act as a single array used to transmit a single acoustic beam. The transmit focal point 2104, which is the center of focal area 2106, can be adjusted by changing the transmit time delay profile. The pathways of ultrasonic energy between each element in transmit aperture 2100 and focal point 2204 is shown by the broken dashed lines 2102. An example of a time delay profile is shown in FIG. 21 B. The time delay profile 2120 shows individual signal time lines, such as time line 2122. Signals such as signal 2124 illustrate the relative time delays for each of the elements in transmit aperture 2100 of FIG. 21A.

FIG. 22 is an illustration of a focal area of a focused ultrasonic beam, according to embodiments of the disclosure. Focal point 2204 is located within focal area 2206. A single ultrasonic beam is generated by a transmit aperture made up of a number of transducer elements (not shown). Pathway 2202 illustrates the pathway between a transducer element and focal point 2204. The size of the focal area 2206 is defined by the depth of field 2230, and by the beam width 2232. If the pressure wave is to be concentrated in a smaller area and the transmit beam is to have better resolution, a larger transmit aperture should be used. If the pressure wave is to cover a larger area, then a smaller transmit aperture should be used. Typically, the focal area can be defined as minus 3 dB of the transmit acoustic intensity at the focal point. Both the depth of field and beam width can be controlled by the number of transducer elements use in a given array. In general, larger numbers of elements generate a narrower beam width and a shorter depth of field. The time delay profile can be symmetrical or asymmetrical. A symmetric time delay profile, as shown in the example of FIG. 21 B, generates a focal point perpendicular to the transducer. An asymmetrical profile steers the transmit beam to a desired area. Single beam focusing allows very tightly focused energy delivery to a small target area. Single beam focusing techniques are relatively simple and are widely used in the conventional ultrasound imaging applications. The GE Logiq 7 made by General Electric of Fairfield, Conn., is an example of a linear array which is suitable for some applications. Besides controlling the focal area size, the number of elements used in a transmit array can be designed so as to improve the signal to noise ratio of the received signals.

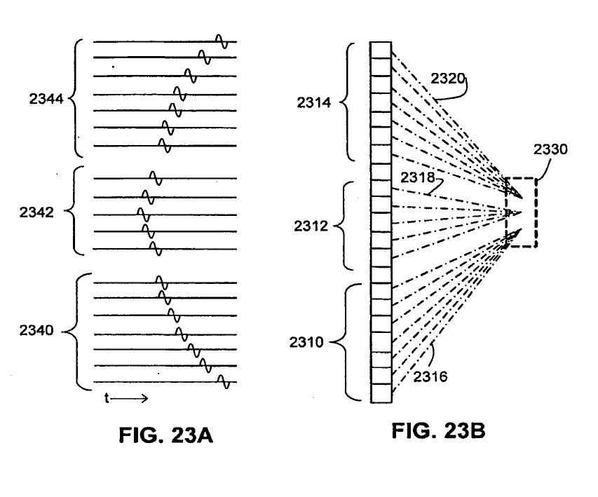

FIGS. 23A-B are of a multiple beam focusing array and associated time delay profile, according to embodiments of the disclosure. Multiple beams generate pressure waves in a larger area than if all the elements were used to focus at that spot. This operation is used when diffraction limited resolution is too tight. In the example shown in FIG. 23B, the transducer elements are divided into groups 2310, 2312 and 2314 which are used to generate beams 2316, 2318 and 2320, respectively. The beams create focal area 2330. While only a small number of individual elements are shown in each group in FIG. 23, depending on the application, other numbers of elements can be arranged in different numbers of groups. For example, a 128-element transducer can be divided into 3 or more groups of transmit apertures. As shown in FIG. 23A, each group of elements has its own transmit time delay profile. Group 2310 uses profile 2340, group 2312 uses profile 2342 and group 2314 uses profile 2344. FIG. 24 shows a multi-beam focal area in greater detail, according to embodiments of the disclosure. As shown, focal area 2330 is made up of three smaller focal areas 2410, 2412 and 2414, which are generated by groups 2314, 2312 and 2310, respectively, and by beams 2320, 2318 and 2316, respectively, all shown in FIG. 238. As described above, the time delay profile can be symmetrical or asymmetrical to generate a straight or steered beam. Multiple beam focusing allows the ultrasound energy to be spread over a larger area than single beam focusing. Manipulation of the beam steering angle also allows the control of the focus area not possible with a single beam focusing setup. The focusing on transmit can be fixed or be variable controlled by software. It is preferable when using multiple beam focusing to use transducer elements that have a relatively large acceptance angle. In practice, for many applications, the beam acceptance angle sets another limit on the number of elements used to focus on transmit or receive.

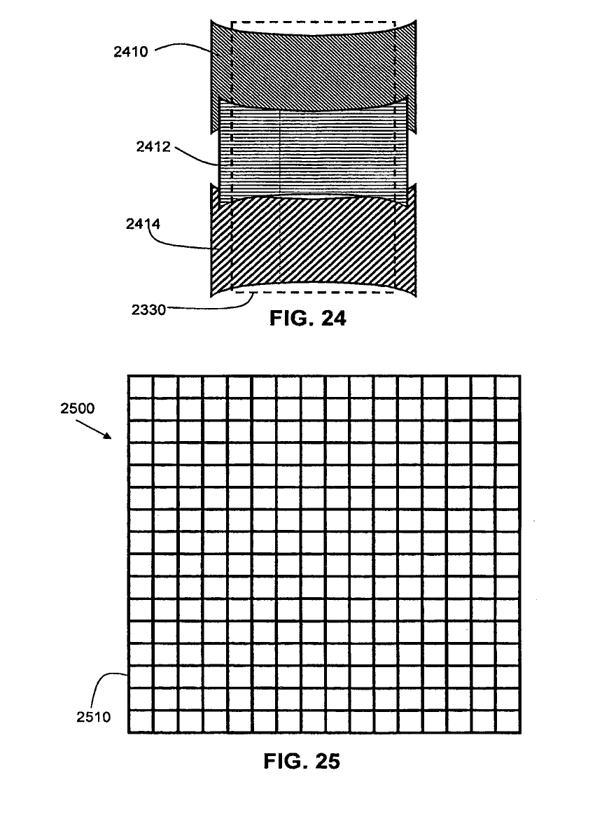

The arrays or transmit apertures shown and described above with respect to FIGS. 21A and 23B are usually flat and use a lens to focus in the direction perpendicular to the transducer, the so called “out of plane” focus direction. FIG. 25 is a two-dimensional transducer array, according to embodiments of the disclosure. 2D array 2500 is shown made up of individual transducer elements 2510. With separation in two dimensions, 2D arrays such as array 2500 can focus, electronically, both in transmit and receive and in two dimensions, hence getting a 3D volumetric image. Advantageously, 2D array transducers can be used to generate radiation pressure to mechanically modulate a vessel and change the position of vessel walls, in accordance with the disclosure.

In accordance with other embodiments of the disclosure, the ultrasonic transducers can be made as a single circular disk (flat or concave for focusing) or an annular array (flat or concave to provide some focusing which can be altered by phase or time delays in the various elements in the annular array). Various design parameters should be taken into account, depending upon the particular application. Typically, a narrow-band, lightly damped tuned and high energy output transducer design is preferred. Typical materials for making this type of transducer are PZT-4 or PZT-8 if the interest is in high power applications. If the interest is in efficiency or broad band operation, then many other types of piezoelectric ceramics are used. Single element transducers and annular arrays can have fewer transmit (and receive) channels than conventional linear and phased arrays. For example a 12 ring annular array can have 12 ultrasound pulse transmitters while a typical phased or linear array transducer ultrasound system requires 128 or more transmitters. Therefore annular arrays typically have inherently less electronics, such as radio-frequency electronics, than comparable linear arrays allowing reduced manufacturing costs.

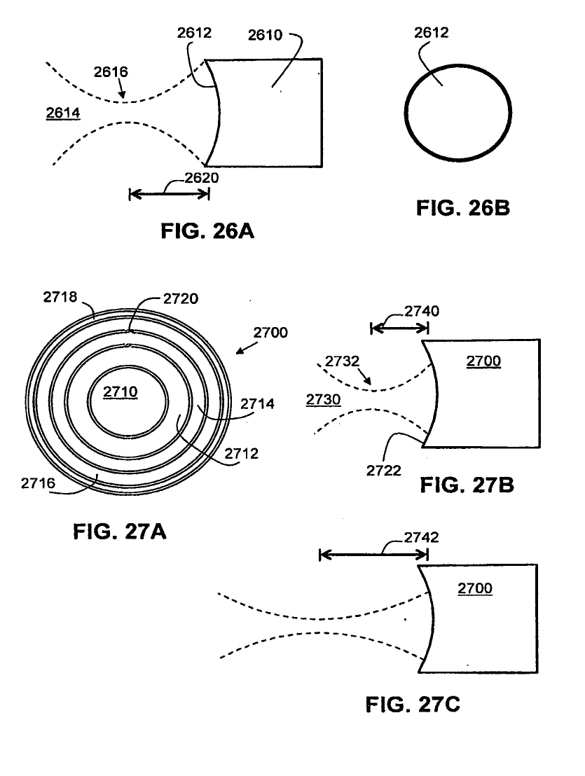

FIGS. 26A-B show a single element transducer having a circular cross-section, according to a preferred embodiment of the disclosure. Referring to FIG. 26A, transducer 2610 has a curved face 2612 and generates energy waves along a pathway 2614, shown between the dashed lines. In this example, the curved element provides focusing, but alternatively focusing can be accomplished using a lens in combination with the transducer element. The focal length 2620 of transducer 2610 is the distance from the face 2612 to the point 2616 in the sound field where the signal with the maximum amplitude is located. FIG. 26B shows the face 2612 of single element transducer 2610. For different clinical applications, single transducers can be designed with a desired focal point. For example, the internal jugular vein is typically located at 20 mm from skin line. Therefore, a single transducer can be designed with 2.25 MHz frequency, 12.7 mm diameter and a focusing radius of curvature of 20 mm. This transducer can generate pressure waves focused approximately at 20 mm below skin line. The advantage of single transducer is that it is simple and inexpensive to design and manufacture. A limitation of a single transducer is that it can typically only be focused at a fixed length and the acoustic beam can typically only be steered mechanically.

FIGS. 27 A-C show an annular array transducer according to embodiments of the disclosure. Referring to FIG. 27A, annular array 2700 comprises an array of circular transducer elements or rings 2710, 2712, 2714, 2716 and 2718. These rings generate a burst comprising parallel trains of one or more electrical pulses. Each pulse train is directed to a respective transducer ring on a respective transmit channel. FIG. 27B illustrates how a focal length shorter then the geometric focal point of transducer 2700 can be achieved by introducing relative delays in the pulse trains. The focal length 2740 is the distance from face 2722 to the point of maximum amplitude 2732 along pathway 2730, shown between the dashed lines. A relatively longer delay applied to the inner transducer rings (e.g., rings 2710 and 2712) result in a wave front which converges closer to the array, i.e., a shallower focal point is affected. FIG. 27C illustrates how applying shorter delays to the inner transducer rings of transducer 2700 affect a greater focal depth 2742. The delays are selected to compensate for the path differences between respective transducer rings and the targeted focal point. Thus, focal depth can be varied by adjusting the relative delays between inner and outer transducer rings. Alternatively, the focal length of the transducer 2700 can be changed by transmitting from only certain rings. Referring again to FIG. 27A, transmitting from only inner ring 2710 will result in a relatively short focal length, while transmitting from rings 2710, 2712, and 2714 will result in a longer focal length. Transmitting from all rings can result in an even longer focal length. Compared with a single transducer, the focal point can be adjusted by relatively inexpensive electronic circuits. Note that although only five rings are shown in transducer 2700 typically larger numbers of ring will be provided. For example, for some applications, array 2700 can comprise 12 or more rings. A commercially available annular array such as the 3.5-5 MHz wide angle annular array known as Ultramark4™ manufactured by Philips Medical Systems can be suitable for some applications.

FIGS. 28A-C show a transducer adapter, according to embodiments of the disclosure. Adapter 2806 is preferably used in combination with either a single element transducer or an annular array transducer. Adapter 2806 can overcome certain inherent limitations of such transducers. Specifically, a single element transducer has a fixed focal point; and an annular array transducer can adjust the focal point but cannot steer the acoustic beam electronically. Adapter 2806 includes sealed reservoir 2812, which contains acoustic couplant 2814, such as silicon oil, gel and other suitable acoustic coupling material. Adapter 2806 is shown in contact with tissue boundary 2834 of tissue 2830. Within tissue 2830 is target structure 2834, through which passes ultrasonic energy pathway 2816, shown between the dashed lines. According to preferred embodiments of the disclosure, tissue boundary 2834 is the patient's skin, tissue 2830 is the patient's neck tissue, and target structure 2832 is the internal jugular vein. The front part of adapter 2806 is covered by a thin layer of membrane 2808, such as Mylar film or some material that provides a suitable acoustic impedance match when making contact with tissue boundary 2834, e.g., human skin. The transducer 2810 is immersed in the couplant 2814 and mechanically mounted on the adaptor 2806. As shown in FIG. 28B, adaptor 2806 allows the transducer 2810 to move backward and forward along the direction of the ultrasound beam pathway 2818, shown between the dashed lines. By moving back and forth in the direction of the ultrasound beam pathway, an adjustment of the focal area below the tissue boundary to overlap with target structure 2832 is achieved. In this way, adapter 2806 can be used with a single element transducer to overcome the limitation of fixed focal length.

As shown in FIG. 28C, adapter 2806 can also allow transducer 2810 to be tilted away from the position which is perpendicular to the tissue boundary 2834. By allowing these degrees of movements, the adapter can effectively be used with a single element transducer or an annular array transducer to steer the ultrasound beam pathway 2820, shown between the dashed lines, to focus the focal area on target structure 2832.

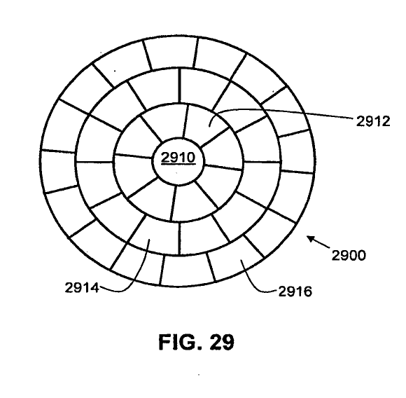

FIG. 29 is a ring array transducer, according to embodiments of the disclosure, ring array transducer 2900 includes 37 transducer elements such as center element 2910, and elements 2912, 2914 and 2916. Ring array transducer 2900 can focus ultrasound energy and scan a volume in front of the transducer. The individual elements are preferably individually addressable and in the phase array examples shown and described above with respect to FIGS. 21A, 23B and 25. Although the example shown in FIG. 29 has only 37 elements, other numbers and arrangements can be used depending on the particular application. For example, a ring with a 2 cm diameter can have over it many 1 mm by 1 mm elements (about 120) that can be all individually addressed as in the phase array above and properly operated, it can deliver a performance comparable to a full 20 array with the same diameter as the ring.

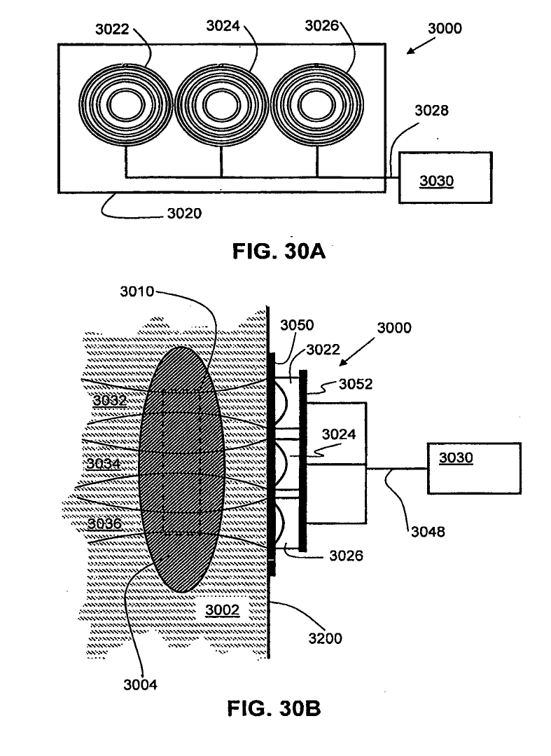

The single transducer and annular array transducer as shown and described above with respect to FIGS. 26, 27 and 29 can also be grouped together. FIGS. 30A-B show a grouped arrangement of annular transducers, according to embodiments of the disclosure. As shown in FIG. 30A, three annular array transducers 3022, 3024 and 3026 are grouped together and mounted on rigid electronic circuit board 3020 to form array group 3000. Coax cable 3028 connects between the transducer circuit board 3020 and transmit pulse power amplifier 3030. In this example, each annular transducer 3022, 3024 and 3026 in array group 3000 is a 12-ring annular array transducer. Thus, array group 3000 has 36 transmit wires placed on circuit board 3020.

FIG. 30B is an alternative embodiment wherein transducers 3022, 3024 and 3026, which can be either annular array transducers or single element transducers, are bonded to matching layer 3050 and to flex circuit 3052 instead of a rigid circuit board. Matching layer 3050 provides acoustic impedance matching between the transducers 3022, 3024 and 3026 and tissue 3002. This embodiment allows for a flexible assembly that can easily conform to human body allowing for a better acoustic interface with human skin. As shown in FIG. 30B, array group 3000 is placed in contact with tissue boundary 3200, which is the patient's skin in some applications, to transmit ultrasonic energy into tissue 3002, and specifically into target structure 3004. Focal area 3010 is shown where the greatest concentration of ultrasonic energy is located due to the focusing of ultrasonic pathways 3032, 3034 and 3036. According to an alternative embodiment, each annular transducer 3022, 3024 and 3026 is a single element transducer instead of an annular array. Using single element transducers greatly simplifies the wiring and circuitry requirements of the array group 3000.

Unlike a single transducer or a single annular array configuration which generates a relatively sharp transmit focus at the target area, the grouped single transducers or annular transducers can deliver ultrasound pressure wave to a larger area, as shown in focal area 3010 in FIG. 30B. Thus, the array group arrangement such as shown in FIGS. 30A and 30B can provide larger focal areas similar to the configurations shown and described above with respect to FIGS. 21A, 23B and 25, but with using much simpler electronic circuitry and smaller size. For some applications, the mechanical adaptor described with respect to FIGS. 28A-C can be used in order to adjust acoustic focal point to a desired location. In designing configurations to meet a particular application, the loss of localization of the region of interaction between the light and the acoustic energy should be taken into account.

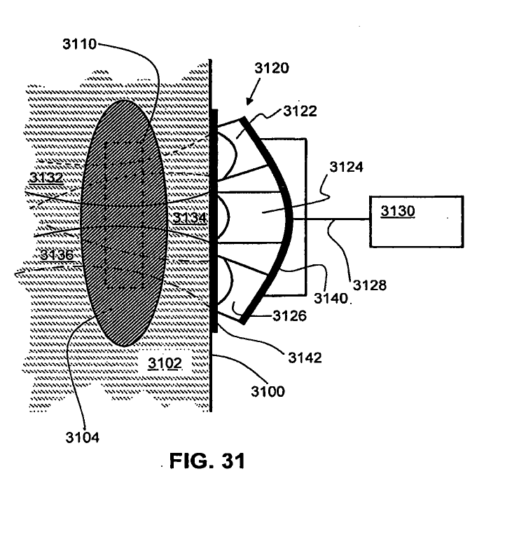

FIG. 31 is a transducer array group having non-parallel transducers, according to embodiments of the disclosure. Array group 3120 is similar to the arrangements shown in FIGS. 30A and 30B. Transducers 3122, 3124 and 3126 can each be either annular array transducers or single element transducers. Each of the transducers is connected to cable 3128 via flex circuit 3140 to transmit pulse power amplifier 3130. Transducers 3122, 3124 and 3126 are mounted on matching layer 3142 which is engaged with tissue boundary 3100 which is typically the patient's skin. Within tissue 3102 is target structure 3104 which is typically a blood vessel such as the patient's internal jugular vein. According to this embodiment, in order to focus the ultrasonic energy in focal area 3110, two of the three transducers, 3122 and 3126, are tilted toward the focal area 3110 such that ultrasonic energy pathways 3132, 3134 and 3136 converge on focal area 3110. To accomplish the tilting, preferably, adapters such as shown and described with respect to FIGS. 28A-C are used. Unlike the arrays as shown and described with respect to FIGS. 21A, 23B and 25, grouped single transducers and, grouped annular transducers, such as shown and described in FIGS. 30A-B and 31, are not capable of electronically steering the transmit energy in a fashion similar to that of the linear multiple beam system. Note that although array groups 3000 and 3120 in FIGS. 30A-B and 31 only have three transducers, or annular arrays, in general, other numbers of transducers or annular arrays can be used depending upon the particular application. For example, if a smaller focal area is suitable, array groups having only two transducers or annular arrays can be used. Likewise, if a larger focal area is suitable, or greater signal to noise ratio is required, then four, five or more single element transducers or annular arrays can be used.

According to alternative embodiments of the disclosure, mechanical vibration generators can provide low frequency, large displacement of a target structure instead of ultrasonic transducers. These techniques can be particularly useful for inducing blood volume changes in target structures close to the skin, such as jugular vein and carotid artery.

Examples of mechanical vibrator technology that can be used in connection with the present disclosure are cell phone vibrators, solenoids, cam followers, slider cranks and other mechanisms. Cell phone vibrators offer a cheap OEM solution providing relatively small displacements at relatively high frequencies. Solenoids are capable of applying large forces over large displacements. Slider cranks and cam followers provide an almost limitless variation of displacement, frequency and force output, but typically have multiple moving parts. The mechanical vibrator can be small and low profile such as those found in a mobile phone. Some of low profile motors have a dimension of 5 mm high, 6 mm wide, and 15 mm long and weighs approximately 19, which can be easily integrated with optical sensor assembly.

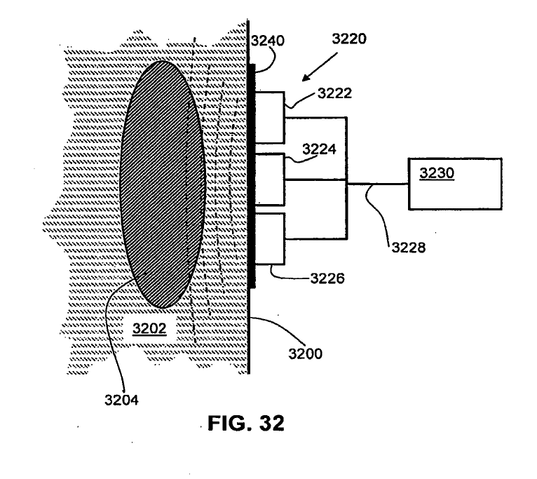

FIG. 32 is a mechanical vibrator arrangement according to embodiments of the disclosure. Vibrator 3220 comprises three mechanical vibrators 3222, 3224 and 3226, which can be cell phone vibrators or other types of vibrators as described above. Each of the mechanical vibrators is connected via cable 3228 to transmit pulse power amplifier 3130. Vibrators 3222, 3224 and 3226 can be mounted on a flexible circuit or other compliant bio-compatible material that enables suitable engagement with tissue boundary 3200, which is typically the patient's skin. Within tissue 3202 is target structure 3204. Vibrational energy passed through tissue 3202 to target structure 3204 where the energy induces a change is the shape of structure 3204. In the case where structure 3204 is a blood vessel, the vibrational energy induces changes in the blood flow which can be detected with the optical systems described herein.

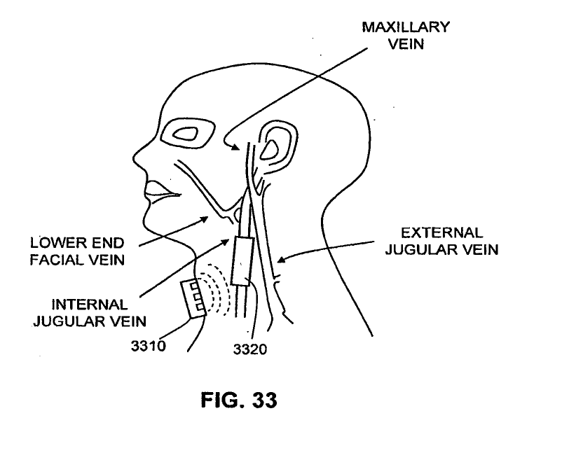

FIG. 33 shows placement of a vibrator group on the skin of a patient, according to embodiments of the disclosure. According to embodiments described above, the ultrasonic transducers, arrays and groups shown and described are generally placed in close proximity to the launch optics and collection optics (or optical transmitters and receivers) since the ultrasonic energy can be focused in an area relatively close to the transducers. However, with mechanical vibrator arrangements, the vibrator and launch/collector optics can be separated by greater distances in some applications. As shown in FIG. 33, vibrator 3310 is located at one location on the neck of the patient, while launch optics and collection optics pad 3320 is located just above the target structure, in this case the internal jugular vein.

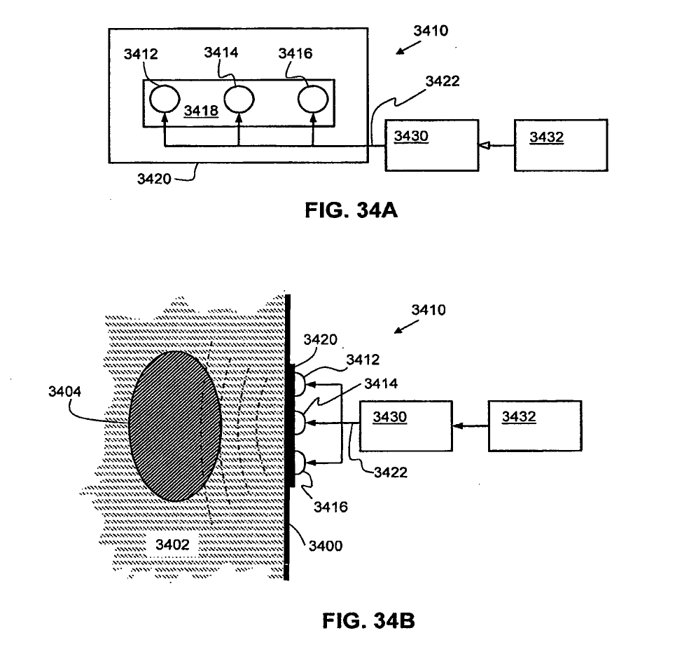

FIGS. 34A-B show an audio loudspeaker arrangement for generating vibrational energy in target structures, according to embodiments of the disclosure. Audio loudspeaker system 3410 includes audio loudspeaker transducers 3412, 3414 and 3416 are mounted on bracket 3418, which is coupled with launch optics and collector optics (not shown), all mounted on mechanical plate 3420. The transducers 3412, 3414 and 3416 receive signals from audio amplifier 3430 via cable 3422. Audio amplifier 3430, in turn, is driven by frequency generator 3432.

As shown in FIG. 34B, loudspeaker system 3420 interfaces with human skin 3400 and produces audio waveforms, which are converted to vibrational energy traveling in tissues 3402 and through target structure 3404. Audio amplifier 3430 controls the frequency of vibration and is preferably a variable frequency and amplitude audio amplifier operating at 5 Hz to 20 KHz ranges. The mechanical vibration generated by loudspeaker system 3410 is applied to the complex medium of living body tissues 3402 to induce vibrations in the target structure 3404. The target structure 3404 can be a large blood vessel such as the internal or external jugular vein or carotid artery. These relatively large blood vessels have a larger displacement compared with high frequency focused ultrasound at 1 MHz to 10 MHz range. Audio frequency vibration can create larger body organ displacements benefiting optical signal detection by generating a higher signal to noise ratio for some applications. Additionally, low frequency vibration waves travel longer distances due to less attenuation in the human tissue. This is particularly useful for certain applications, such as fetal monitoring applications. Finally, because audio waves are less focused than ultrasonic waves, there is a reduced potential risk for negative biological effects on human tissues.

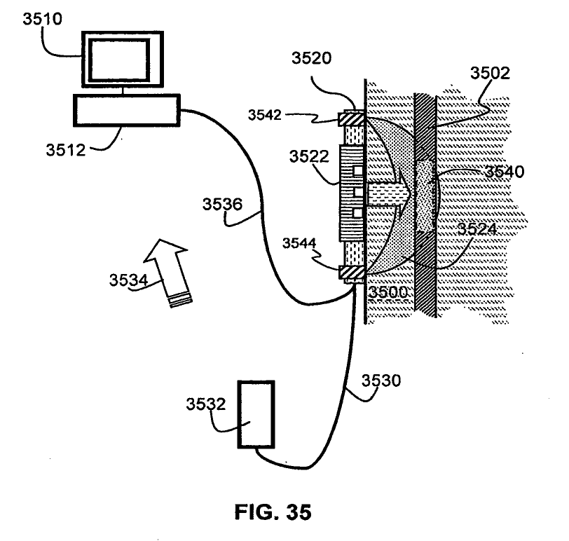

FIG. 35 shows a system for making relative measurements relating to blood oxygenation according to an embodiment of the disclosure. As shown in FIG. 35, the system includes a patch sensor 3520. Sensor 3520 includes one or more electromagnetic radiation transmitters 3542, one or more electromagnetic radiation detectors 3544, and at least one acoustic transducer system 3522. Transmitters 3542 preferably transmit continuous wave energy. Acoustic transducer system 3522 can be one or more of the ultrasonic, vibrational, or audio systems as shown and described above with respect to FIGS. 21, 23-32 and 35. The acoustic energy from system 3522 travels through the patient's body 3500, such as tissues of the neck, to the focal area 3540 which substantially includes target blood vessel 3502. The electromagnetic radiation from transmitters 3542 transmits into the body 3500, including target blood vessel 3502, in which measurements related to blood oxygen saturation are taken. The target blood vessel 3502 can be more than 1 cm below the surface of the skin (or other tissue boundary) at the location where patch 3520 is engaged, and in many cases, such as where the target blood vessel is the internal jugular vein in an adult patient, vessel 3502 is typically about 1.5 to 2 cm below the skin. The crescent-shaped pathway 3524 of the radiation transmitted by transmitters 3542 is scattered through tissues of body 3500 and collected by the detectors 3544 as shown. According to some embodiments of the disclosure, the transmitters and detectors are configured, arranged and/or positioned such that two or more pathways include at least two different penetration depths, for example, by providing two different transmitter/detector pair separation distances (not shown). As shown in FIG. 35, pathway 3524 includes blood vessel 3502.

Optical fiber cables or electronic wire cables 3530 and 3536 connect the patch 3520 to either a main station box 3512, or to a portable unit 3532 which sends out data through wireless communication to station box 3512 as illustrated by arrow 3534. In communication with station box 3512, display 3510 shows both time course trend and digits of oxygen saturation in the blood vessel(s) of interest, e.g., the internal jugular vein and/or carotid artery, as well as oxygen consumption rate. Portable unit 3532 is preferably dimensioned and sized such that the patient can carry the portable box for extended periods.

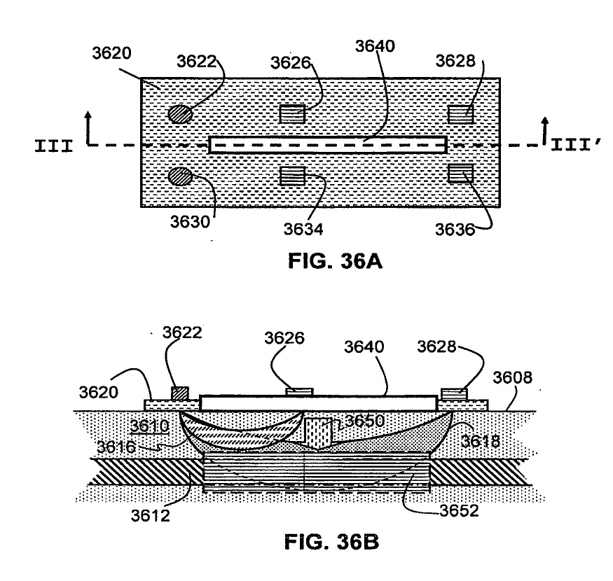

FIGS. 36A and 36B show a sensor/transducer unit according to embodiments of the disclosure. FIG. 36A shows sensor/transducer unit 3620 that includes two electromagnetic transmitters 3622 and 3630, four electromagnetic receivers 3626, 3628, 3634 and 3636. Transmitters 3622 and 3630 preferably transmit continuous wave energy. Additionally, sensor/transducer unit 3620 includes acoustic transducer unit 3640, which can be similar or identical to the ultrasonic transducer 206 as shown and described, for example, with respect to FIGS. 2A and 2B, or one of the ultrasonic, vibrational, or audio systems as shown and described above with respect to FIGS. 21, 23-32 and 35. Transducer unit 3640 is typically coupled to a separate signal source (not shown). FIG. 36B is a cross-section of the sensor/transducer unit 3620 along III-III′ in FIG. 36A. As shown, the transmitter-receiver pairs 3622-3626 and 3622-3628 generate electromagnetic radiation pathways 3616 and 3618, respectively. Transducer unit 3640 is positioned as shown to be used to both image the underlying tissues 3610, for example to precisely locate blood vessel 3612, and to induce changes so as to modulate the blood vessel 3612 to provide for more accurate measurement, as described in detail elsewhere herein. The spacing between the transmitter/receiver pairs 3622-3626 and 3622-3628 should be chosen such that the depth of the resulting radiation pathway is appropriate for the particular application. For example, in some cases where sensor/transducer unit 3620 is placed on the skin of the neck, and the shallower radiation pathway between transmitter/receiver pair 3622-3626 is to include the superficial tissues of the neck but not the internal jugular vein, a spacing of about 2 cm has been found suitable, and the deeper pathway between transmitter/receiver pair 3622-3628 is to include the superficial tissues as well as the internal jugular vein, a spacing of about 5 cm has been found suitable.

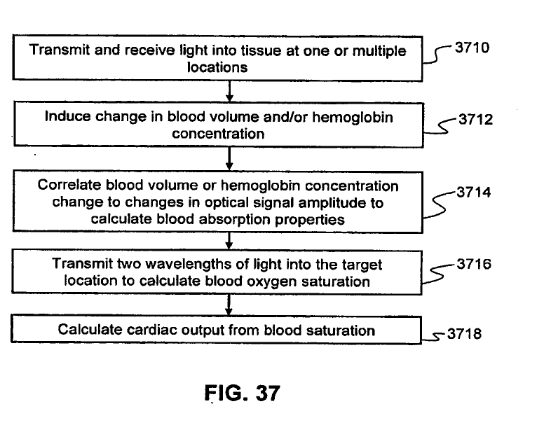

FIG. 37 is a flowchart illustrating several steps relating to measuring cardiac output according to embodiments of the disclosure. In step 3710, electromagnetic radiation is transmitted into the patient's tissues at one or more locations and back scattered light is detected or received at one or more locations. The radiation is preferably transmitted and received using the systems and apparatus as shown and described above with respect to FIGS. 35-36. The radiation preferably contains at least one wavelength ranging from 600 nm to 900 nm. The radiation is transmitted into a target area within a target structure, which in some applications can be the patient's internal jugular vein. According to some embodiments, for example, as shown and described with respect to FIGS. 36A-B, two or more different pathways having different depths are transmitted and received by two or more transmitter/receiver pairs.

In step 3712, an ultrasound transducer or array is used to generate pressure in the target area within the target structure, such as at the wall of the patient's internal jugular vein. The pressure will generate changes in the local blood volume as well as the hemoglobin concentration. Ultrasound arrangements as shown and described with respect to FIGS. 21A, 23B, 25-31 can be used, or alternatively other methods of inducing changes in the blood volume and/or hemoglobin concentration can be used such as the vibrational or audio systems shown and described with respect to FIGS. 31-33 can be used. The induced changes are preferably periodic at frequency of from 5 Hz to 100 Hz, or from 100 Hz to a 2-3 KHz.

In step 3714, the induced changes in the blood volume generated by ultrasound pressure wave or other methods will change the amplitude of the signal detected by the electromagnetic sensors/receivers. These changes in the detected signals may be correlated to the local blood volume changes through the following equation.

δU=WμₐδV (1)

Where δU is the change in optical signal, μₐ is the absorption property of blood, δV is the volume change caused by ultrasound pressure waves or other means, W is the photon probability density at the target location. W can be calculated through the photon diffusion equation:

$$- D {∇}^{2} Φ ( r , t ) + v {μ}_{a} Φ ( r , t ) + \frac{\partial Φ ( r , t )}{\partial t} = vS ( r , t ) \qquad ( 2 )$$

Where D is the diffusion constant, v is the speed of light, μₐ is the absorption coefficient of medium to the light.

$$W = \frac{1}{4 π D ( \vec{r} - {\vec{r}}_{s} )} {e}^{i k ( \vec{r} - {\vec{r}}_{s} )} \cdot \frac{1}{4 π D ( {\vec{r}}_{d} - \vec{r} )} {e}^{i k ( {\vec{r}}_{d} - \vec{r} )} \qquad ( 3 )$$

The blood absorption properties can be then calculated through equation (1).

In step 3716, electromagnetic radiation at two or more different wavelengths between 600 nm to 900 nm are transmitted through the target area in the target structure to obtain the absorption properties of the target structure, for example, patient's internal jugular vein, at each wavelengths and to calculate blood oxygen saturation in the target area using the following equations.

$$\begin{matrix}{μ}_{a , IJ}^{λ} = {C}_{Hb} {ɛ}_{Hb}^{λ} + {C}_{HbO} {ɛ}_{HBO}^{λ} & ( 4 ) \\ S {O}_{2} = \frac{{C}_{HbO}}{{C}_{HbO} + {C}_{Hb}} \% & ( 5 )\end{matrix}$$

In step 3718, the oxygen saturation in the target structure is then used to calculate cardiac output using the relationship:

$$CardiacOutput = \frac{OxygenConsumption}{( {S}_{a} {O}_{2} - {S}_{IJ} {O}_{2} ) A} \qquad ( 6 )$$

Where SaO2 is the arterial blood oxygen saturation measured through standard pulse oximetry.

According to some embodiments, as mentioned, electromagnetic radiation is transmitted through a second pathway having a shallower depth. Measurements from the second pathway are used to measure the average tissue scattering and absorption properties of the superficial layer above the blood vessel (e.g., the internal jugular vein). Preferably, the shallower or shallowest pathway should substantially exclude the blood vessel of interest (in many cases, the internal jugular vein). In order to provide more accurate calculations for W1, as described below, spatial locations within the blood vessel should have less than about 20% photon probability. Even more preferably, spatial locations within the blood vessel should have less than about 5% probability for photons traveling between the transmitter and receiver pair for the shallowest pathway. Finally, it has been found that in order to further increase the practical applicability and further increase the accuracy for calculations for W1, the photon probability should preferably be less than about 1%.

The measurements from the transmitter-receiver or source-detector pair having the shallower depth are used to calculate the probability of photon distribution inside depth from surface to z₁ (z₁ is typically 2 cm) which is W₁.

$${W}_{1} = {\int }_{0}^{{z}_{1}} [ \begin{matrix}{\int }_{∞} \int \frac{1}{4 π D ( \vec{r} - {\vec{r}}_{s} )} {e}^{i k ( \vec{r} - {\vec{r}}_{s} )} \cdot \\ \frac{1}{4 π D ( {\vec{r}}_{d} - \vec{r} )} {e}^{i k ( {\vec{r}}_{d} - \vec{r} )} d x d y\end{matrix} ] d z \qquad ( 7 )$$

The measurements from the electromagnetic pathway having the greater depth are used to calculate the photon probability distribution from depth z₁ to z₂, which is W₂:

$${W}_{2} = {\int }_{{z}_{t}}^{{z}_{2}} [ \begin{matrix}{\int }_{∞} \int \frac{1}{4 π D ( \vec{r} - {\vec{r}}_{s} )} {e}^{i k ( \vec{r} - {\vec{r}}_{s} )} \cdot \\ \frac{1}{4 π D ( {r}_{d} - \vec{r} )} {e}^{i k ( {\vec{r}}_{d} - \vec{r} )} d x d y\end{matrix} ] d z \qquad ( 8 )$$

Where rₛ the position of light source, r_{d} is the position of detector, and r is the position of a certain position inside medium. K is the wave vector which can be derived from the photon diffusion equation, D is the diffusion constant of medium.

From W₁ and W₂ and the absorption property of tissue at depth from z₁ to z₂ can be derived from equation:

$${μ}_{a , IJ} = \frac{{\overline{μ}}_{a , depth 2} - {W}_{1} \cdot {μ}_{a , depth 1}}{{W}_{2}} \qquad ( 9 )$$

Note that while the present and several of the foregoing embodiments have been described using the example of blood oxygen saturation and cardiac output, the disclosure is also applicable to monitor other parameters relating to the patient's blood. For example, blood pH can be monitored using met-hemoglobin as a target chromophore, as is described in further detail above. Another example is monitoring water and/or lipid in the blood, using radiation wavelengths which are selected to be suitable for the particular chromophore application.

According to an alternative embodiment of the disclosure, the SₐO₂ can be measured without using conventional means, such as a standard pulse oximeter. According to this embodiment, SₐO₂ is measured through the same patch sensor as shown in FIG. 23 as described above, the amplitudes of the optical signals, especially the source-detector pair with the largest separation, are modulated by the pulsation by the major artery which is adjacent to the internal jugular vein, i.e., the carotid artery. The amplitudes of such modulated signals at two or more different wavelengths are used to calculate the oxygen saturation of arterial blood, as described above.

Although the above description emphasizes measurement of blood oxygenation for the purpose of determining venous oxygen saturation, cardiac output and pH, the disclosure is not limited to such applications. The technique described herein can be adapted to selectively probe tissues within the body to measure the level of a particular target chromophore within those tissues and derive diagnostic information about the tissue from the measurement. These measurements can be made in a manner which is accurate, reproducible, precise, fast, operator independent, easy to use, continuous, cost effective, and substantially free of increased mortality and morbidity. Embodiments of the present disclosure allow measurements that used to be made in a highly invasive manner to be made in a non-invasive or minimally invasive manner. Applications of the technique include measuring the health of a transplanted organ to check for signs of rejection, measuring the perfusion of a skin graft in, for example, a burn victim, to determine the health of the graft, potential ambulatory monitoring of high-risk cardiovascular patients, and ambulatory monitoring of high-risk pregnancies.

Although several of the foregoing embodiments have been described using the internal jugular vein as a target structure for monitoring, there are a number of other target structures within the body that are suitable for blood oxygen monitor using embodiments of the present disclosure. Several representative example applications will now be described. The exterior jugular vein can be monitored transdermally as shown and described above with respect to FIG. 13. The right subclavian vein, superior vena cava, pulmonary artery and other major blood vessels may be monitored as shown and described above with respect to FIG. 14. Neonatal blood oxygenation can be monitored as shown and described above with respect to FIGS. 18A-18C, and 19. Fetal monitoring can be provided as shown and described above with respect to FIG. 20.

The techniques described are not limited to the hospital or medical office setting. Embodiments of the disclosure could be made portable and simple to use by virtue of its use of rugged telecom components and low power-consumption devices which could in turn allow its use in ambulances. Embodiments of the disclosure may be useful for real-time monitoring of personnel in high risk situations. For example, rescue workers in chemical plants responding to emergencies or firemen in burning buildings could be monitored remotely for signs of physical distress. Military personnel with ambulatory versions of the sensors could be monitored on the battlefield, and portable versions of the device could be used for first-responder battlefield triage.

While the above is a complete description of the preferred embodiment of the present disclosure, it is possible to use various alternatives, modifications and equivalents. Therefore, the scope of the present disclosure should be determined not with reference to the above description but should, instead, be determined with reference to the appended claims, along with their full scope of equivalents. In the claims that follow, the indefinite article “A”, or “An” refers to a quantity of one or more of the item following the article, except where expressly stated otherwise. The appended claims are not to be interpreted as including means-plus-function limitations, unless such a limitation is explicitly recited in a given claim using the phrase “means for.”

1. A method for monitoring one or more parameters relating to blood of a patient.
